(19) United States
(12) Patent Application Publication
(10) Pub. No.: US 2021/0161868 A1
(43) Pub. Date: Jun. 3, 2021

(54) ANTI-INFLAMMATORY THERAPY IN ARRHYTHMOGENIC CARDIOMYOPATHY (ACM)

(71) Applicant: Beth Israel Deaconess Medical Center, Inc., Boston, MA (US)

(72) Inventor: Jeffrey E. Saffitz, Waban, MA (US)

(21) Appl. No.: 17/050,259

(22) PCT Filed: Apr. 25, 2019
(86) PCT No.: PCT/US2019/029151

§ 371 Date: Oct. 23, 2020

Related U.S. Application Data
(60) Provisional application No. 62/662,273, filed on Apr. 25, 2018.

(51) Int. Cl. A61K 31/436; A61K 31/277; A61P 9/06

(52) U.S. Cl. CPC A61K 31/436; A61P 9/06; A61K 31/277

(57) ABSTRACT

Described herein are, inter alia, methods for treating arrhythmogenic cardiomyopathy (ACM) using anti-inflammatory agents that target nuclear factor-kappa-B (NFkB).

### CLAIM OF PRIORITY

This application claims the benefit of U.S. patent application Ser. No. 62/662,273, filed on Apr. 25, 2018. The entire contents of the foregoing are hereby incorporated by reference.

### FEDERALLY SPONSORED RESEARCH OR DEVELOPMENT

This invention was made with Government support under Grant No. HL116906 awarded by the National Institutes of Health. The Government has certain rights in the invention.

### TECHNICAL FIELD

Described herein are, inter alia, methods for treating arrhythmogenic cardiomyopathy (ACM) using anti-inflammatory agents that target nuclear factor-kappa-B (NFκB).

### BACKGROUND

Arrhythmogenic cardiomyopathy (ACM), also known as arrhythmogenic right ventricular cardiomyopathy in particular (ARVC), is associated with a high frequency of arrhythmias and sudden cardiac death (Marcus et al., Circulation 1982; 65:384-98; Thiene et al., N Engl J Med 1988; 318:129-33; Dalal et al., Circulation 2005; 112:3823-32), ACM is a familial non-ischemic heart muscle disease that causes sudden death in the young and especially in athletes.¹⁻³ Heightened risk in athletes underscores increasing awareness that intense exercise accelerates disease penetrance, and increases arrhythmic risk and adverse cardiac events in subjects who harbor ACM disease alleles.^{4,5} Currently, the only effective treatment is an Implantable Cardioverter Defibrillator (ICD). Mutations in genes encoding desmosomal proteins (including desmoplakin, plakoglobin, plakophilin 2, desmocollin 2, and desmoglein 2) have been identified in approximately 60% of patients with ARVC (te Riele et al., J Cardiovasc Magn Reson. 2014; 16:50).

### SUMMARY

As shown herein, an innate immune response in cardiac myocytes drives the ACM disease phenotype. This mechanism is greatly intensified by exercise.

Thus, provided herein are methods for treating a subject with arrhythmogenic cardiomyopathy (ACM). The methods include identifying a subject as having or at risk of developing ACM; and administering to the subject a therapeutically effective amount of an inhibitor of NFκB signaling.

In some embodiments, the inhibitor of NFκKB signaling is selected from the group consisting of DNA binding inhibitors that inhibit the binding between NFκB and DNA; inhibitors of post-translational modifications on NFκB including a p65 acetylation inhibitor; translocation inhibitors that prevents NFκB from translocating to the nucleus; IκB degradation inhibitors that prevents ubiquitinated IκB from being degraded; IKK inhibitors that prevent the phosphorylation of IκB bound to NFκB.

In some embodiments, the inhibitor of NFκB signaling is an IKK inhibitor that prevents the phosphorylation of Iκb bound to NFκB.

In some embodiments, the IKK inhibitor is an ATP analog, an allosteric modulator, or an agent interfering with the kinase activation loops.

In some embodiments, the IKK inhibitor is selected from the group consisting of β-carboline, SPC-839, BMS-345541, SAR-113945, and Bay 11-7082.

In some embodiments, the inhibitor of NFκB signaling is selected from the group consisting of Bay 11-7082; Bithionol; Bortezomib; Cantharidin; Chromomycin A3; Daunorubicinum; Digitoxin; Ectinascidin 743; Emetine; Fluorosalan; Manidipine hydrochloride; Narasin; Lestaurtinib; Ouabain; Rapamycin; Sorafenib tosylate; Sunitinib malate; Tioconazole; Tribromsalan; Triclabendazolum; and Zafirlukast. In some embodiments, the inhibitor of NFκB signaling is Bay 11-7082. In some embodiments, the inhibitor of NFκB signaling is rapamycin.

In some embodiments, the method further comprises one or more of recommending or advising the subject to avoid strenuous or intense physical activity or exercise; recommending or prescribing or administering one or more Singh Vaughan Williams class II antiarryhthmics (beta blockers) such as propranolol, esmolol, timolol, metoprolol, or atenolol; recommending or prescribing or administering one or more class III anti-arrhythmics (K-channel blockers) such as amiodarone, sotalol, ibutilide, dofetilide, dronedarone or E-4031; recommending or performing cardiac ablation; or recommending or implanting an implantable cardiac defibrillator (ICD).

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, inc5deluding definitions, will control.

Other features and advantages of the invention will be apparent from the following detailed description and figures, and from the claims.

### DESCRIPTION OF DRAWINGS

FIGS. 1A-1B. Reversal of ACM features by Bay 11-7082 in neonatal rat ventricular myocytes (NRVMs) expressing a deletion mutation in the gene for plakoglobin (JUP^{2157del2}). A. Representative confocal immunofluorescence images from control (non-transfected) NRVMs, and NRVMs expressing JUP^{2157del2} in the absence or presence of Bay 11-7082. Arrows show localization of immunoreactive signal at the cell surface. The normal distribution of N-cadherin in all cells is shown as a positive control. Untreated JUP ^{2157del2} cells showed abnormal distribution of plakoglobin, Cx43 and GSK3β. The amount of signal for plakoglobin and Cx43 at cell-cell junctions was greatly reduced in these cells whereas signal for GSK3β, which normally resides in the cytoplasm, was seen at the cell surface. Asterisks identify apparent nuclear localization of plakoglobin in JUP ^{2157del2} cells. The abnormal distribution of plakoglobin, Cx43 and GSK3β was normalized in JUP ^{2157del2} cells treated with Bay 11-7082. B. TUNEL labeling in control NRVMs, and NRVMs expressing JUP ^{2157del2} in the absence or presence of Bay 11-7082. Representative confocal images show increased TUNEL+nuclei (arrow heads) in cultures of cells expressing JUP ^{2157del2} and normalization after treatment with Bay 11-7082. The graph shows the % apoptotic nuclei in 5 microscopic fields from each condition.

Scale bar=50 μm. * P<0.05 for JUP^{2157del2} cells vs. control; † P<0.05 for treated vs. untreated JUP ^{2157del2} cells.

FIG. 2. Bay 11-7082 reduces cytokines in the culture media in neonatal rat ventricular myocytes (NRVMs) expressing a deletion mutation in the gene for plakoglobin (JUP^{2157del2}). Representative cytokine arrays are shown for control (non-transfected) cells and NRVMs expressing JUP^{2157del2} in the absence or presence of Bay 11-7082. The spots in the upper right and left and lower left corners are reference markers to compare overall exposure levels.

FIGS. 3A-3H. Reversal of ACM disease features in Dsg2^{mut/mut} mice in vivo by inhibition of NFκB signaling with Bay 11-7082. A. Representative short-axism-mode echocardiograms of vehicle-treated wildtype (WT) mice, Dsg2^{mut/mut} mice and Dsg2^{mut/mut} mice treated with Bay 11-7082. B. Group data for % ejection fraction in wildtype (WT) mice, Dsg2^{mut/mut} mice and Dsg2^{mut/mut} mice treated with Bay 11-7082. C. Representative long-axis sections of the hearts stained with Masson trichrome from wildtype (WT) mice, Dsg2^{mut/mut} mice and Dsg2^{mut/mut} mice treated with Bay 11-7082. D. Group data for % of left ventricular area occupied by fibrosis in Masson trichrome stained sections of hearts from wildtype (WT) mice, Dsg2^{mut/mut} mice and Dsg2^{mut/mut} mice treated with Bay 11-7082. E. Representative images showing TUNEL labeling in sections of hearts from wildtype (WT) mice, Dsg2^{mut/mut} mice and Dsg2^{mut/mut} mice treated with Bay 11-7082. F. Group data showing % apoptotic nuclei in TUNEL labeled sections of hearts from wildtype (WT) mice, Dsg2^{mut/mut} mice and Dsg2^{mut/mut} mice treated with Bay 11-7082. G. Representative signal-averaged electrocardiograms from wildtype (WT) mice, Dsg2^{mut/mut} mice and Dsg2^{mut/mut} mice treated with Bay 11-7082. H. Representative confocal images of immunostained hearts from wildtype (WT) mice, Dsg2^{mut/mut} mice and Dsg2^{mut/mut} mice treated with Bay 11-7082. Arrows show localization of immunoreactive signal at the cell surface. The normal distribution of N-cadherin in all cohorts is shown as a positive control. Untreated Dsg2^{mut/mut} mice showed abnormal distribution of plakoglobin, Cx43, GSK3β and SAP97. The amount of signal for plakoglobin, Cx43 and SAP97 at cell-cell junctions was greatly reduced, whereas signal for GSK3β, which normally resides in the cytoplasm, was seen at the cell surface. These abnormal protein distributions were normalized in Dsg2^{mut/mut} mice treated with Bay 11-7082.

FIGS. 4A-4B. Cytokine expression in the hearts of Dsg2^{mut/mut} mice and its attenuation by Bay 11-7082. A. Representative cytokine arrays from hearts of vehicle-treated wildtype (WT) mice, Dsg2^{mut/mut} mice and Dsg2^{mut/mut} mice treated with Bay 11-7082. The spots in the upper right and left and lower left corners were used as reference markers (RBs) to compare overall exposure levels. B. Quantitative data (mean±SEM; n=5) for expression of selected cytokines in hearts of vehicle-treated wildtype (WT) mice, Dsg2^{mut/mut} mice and Dsg2^{mut/mut} mice treated with Bay 11-7082. *P<0.05 compared to WT; † P<0.05 for treated vs. untreated Dsg2^{mut/mut} mice.

FIGS. 5A-5B. Cytokine expression in cardiac myocytes and infiltrating inflammatory cells in hearts of Dsg2^{mut/mut} mice. A. Representative immunoperoxidase stained sections of myocardium from vehicle-treated wildtype (WT) mice, Dsg2^{mut/mut} mice and Dsg2^{mut/mut} mice treated with Bay 11-7082 showing immunoreactive signal distributions for IL-1β, TNFα and MCP1α. Signal intensities for all 3 cytokines was increased in myocardial sections from Dsg2^{mut/mut} mice. Signals for IL-1β and TNFα were seen in both cardiac myocytes and infiltrating inflammatory cells in hearts of Dsg2^{mut/mut} mice. Treatment with Bay 11-7082 reduced signal intensity. B. Immunoperoxidase stained sections of myocardium from Dsg2^{mut/mut} mice showed the presence of both macrophages (CD68+ cells) and T-cells (CD3+ cells) (asterisks). Scale bars=25 μm.

FIGS. 6A-6D. Correlations between cardiac function, myocardial injury and cytokine expression in Dsg2^{mut/mut} mice. A,B. Pearson's correlation between cardiac function (ejection fraction) and myocardial injury (fibrosis and apoptosis) for all Dsg2^{mut/mut} mice treated with Bay 11-7082 (showing responders and non-responders) in panel A, and for all cohorts in panel B. C,D. Correlations between expression levels of LIX (panel C) and OPN (panel D) and ejection fraction for each animal in all cohorts. Data are expressed as mean±SEM; n=5 for vehicle-treated WT and untreated Dsg2^{mut/mut} mice; n=17 for Dsg2^{mut/mut} mice treated with Bay 11-7082.

FIGS. 7A-7E. Correlations between LIX and OPN expression and myocardial fibrosis and apoptosis in all mice (panels A-D), and correlations between expression levels of selected cytokines (E).

FIGS. 8A-8D. Cytokine expression in control and ACM patient derived hiPSC-cardiac myocytes. A. Representative cytokine arrays prepared from cultures of cardiac myocytes derived from a control hiPSC cell line and a line from a patient with a disease causing variant in PKP2. Arrays are shown for cells grown in the absence or presence of Bay 11-7082. The spots in the upper right and left and lower left corners are reference markers (RBs) to compare overall exposure levels. B. Quantitative data (mean±SEM; n=3) for expression of selected cytokines in control cells and PKP2 cells with or without Bay 11-7082. *P<0.05 compared to control cardiac myocytes; † P<0.05 for treated vs. untreated PKP2 cardiac myocytes. C. Representative cytokine arrays prepared from culture media (supernatant) from cardiac myocytes derived from a control hiPSC cell line and a line from a patient with a disease causing variant in PKP2. Arrays are shown for media isolated from cells grown in the absence or presence of Bay 11-7082. The spots in the upper right and left and lower left corners are reference markers (RBs) to compare overall exposure levels. D. Quantitative data (mean±SEM; n=3) for expression of selected cytokines in control cells and PKP2 cells with or without Bay 11-7082. *P<0.05 compared to supernatants from control cardiac myocytes; † P<0.05 for supernatants from treated vs. untreated PKP2 cardiac myocytes.

FIGS. 9A-9B: Effects of the NFκB blocker Bay 11-7082 and rapamycin on neonatal rat ventricular myocytes (NRVMs) expressing a mutant form of plakoglobin. Wildtype cells showed strong immunoreactive signals for the desmosomal protein plakoglobin and the gap junction protein Cx43 at cell-cell junctions, and few if any apoptotic cells (TUNEL+ labeling). In mutant cells, plakoglobin signal was redistributed to the cytoplasm and nuclei. Cx43 signal at junctions was greatly reduced, and many apoptotic cells are shown (arrows). Treatment of mutant cells with Bay 11-7082 (3A) for 24 hours completely reversed these in vitro readouts of the ACM phenotype. Similar effects were seen in ACM cells treated with rapamycin (FIG. 3B), which also blocks NFκB signaling.

FIG. 10. Cytokines produced by control and ACM NRVMs at rest and after mechanical stimulation. Stretching control cells induced an increase only in VEGF, an adaptive physiologic response. By contrast, ACM myocytes showed increased production of TNFα, IL-17, IFγ, IL-6, MIP-1α and others at rest, and greatly increased production after stretch. Cytokines in colored circles have been identified in ACM patients.¹³

FIG. 11. The canonical NFκB pathway. NFκB/Rel (p50/p65) proteins in the cytosol are bound to and inhibited by IκB (inhibitor of κB) proteins. Stimulation of cell surface receptors by cytokines, LPS, antigens, etc., activates an IKK (IκB kinase) complex that phosphorylates IκB proteins, and thereby targets them for ubiquitination and degradation. The resulting free NFκB/Rel complexes are further activated by various post-translational modifications (phosphorylation, acetylation and/or glycosylation, reflecting actions of many regulatory enzymes), and translocate to the nucleus where they combine with other transcription factors to regulate gene expression in the immune response. This diagram is greatly simplified. It does not show multiple modulating factors such as GSK3β-mediated phosphorylation of CREB and its downstream effects on p65 and subsequent activation of the p50/p65 (NFκB/Rel) complex. Black arrows show stimulation of the NFκB pathway and grey lines show sites of inhibition by Bay 11-7082 and sodium salicylate.

### DETAILED DESCRIPTION

Inflammation has been recognized as a feature of ACM for as long as the disease has been known.⁸ First described by autopsy pathologists,⁹ inflammatory infiltrates occur in the hearts of 60 to 88% of ACM patients, and are especially common in ACM patients who died suddenly.^{9,10} It has been suggested that a histologic picture reminiscent of acute myocarditis may reflect an active phase of ACM associated with accelerated disease progresssion,¹¹ but the presence of inflammatory cells in the myocardium in ACM is only part of the story. ACM patients have elevated circulating levels of pro-inflammatory cytokines, and cardiac myocytes themselves produce potent cytokines in ACM.¹² Thus, inflammation in ACM is complex. It involves infiltrating inflammatory cells and activation of an immune response in cardiac myocytes, one or both of which may contribute to disease expression. However, this question has never been rigorously investigated. Immune activation occurs in many heart diseases (ischemia/reperfusion, pressure/volume overload, infections, autoimmunity) but its contribution to tissue injury varies greatly in specific settings. There is a large literature on the potential role of inflammatory cytokines in heart failure, but relatively little work has been done on this question in the cardiomyopathies. Whereas corticosteroid use in Duchenne muscular dystrophy is associated with improved cardiac function and reduced fibrosis,^{13,14} the role of inflammation as a driver of myocardial injury in the non-ischemic cardiomyopathies has not been studied in detail.

Both components of the immune response in ACM likely contribute to disease pathogenesis. The most conspicuous component is infiltration of the myocardium by “professional” cells of the adaptive immune response—lymphocytes and macrophages. Indeed, inflammatory cells can be so abundant in the hearts of ACM patients that the disease may be misdiagnosed as myocarditis.⁵² However, it has never been clear if inflammatory cells accumulate in the heart in ACM only as a reparative response to myocardial damage or if such cells actually promote arrhythmias and/or myocyte injury mediated by immune mechanisms. The second component involves activation of an innate immune response in cardiac myocytes in ACM. How this occurs is unclear although it is known that activation of GSK3β promotes inflammation through NFκB signaling.²¹⁻²⁴ In any event, we show here that cardiac myocytes that express variants in 3 different desmosomal genes known to cause ACM in patients produce and secrete large amounts of diverse chemical mediators of the immune response. Many of these are powerful chemoattractant molecules that likely play an important role in mobilizing bone marrow-derived inflammatory cells to the heart. Cardiac myocytes in ACM also produce powerful pro-inflammatory mediators such as IL-1β and TNFα, both of which are considered primordial cytokines of the innate immune response. This suggests that activation of immune signaling within cardiac myocytes may play an important role in driving the key clinical features of the disease. It also raises the interesting possibility that cytokines made and secreted by cardiac myocytes act in an autocrine fashion to alter ion channel function and promote arrhythmias in ACM. If so, this would add to the traditional view of the role of inflammation in arrhythmogenesis which holds that cardiac ion channel dysfunction is mediated by cytokines produced by lymphocytes and macrophages that infiltrate the heart in myocarditis or other inflammatory heart diseases.⁵³

Glycogen synthase kinase-3β (GSK3β) plays a central role in the pathogenesis of ACM.⁶ A small molecule, SB216763, annotated as an inhibitor of GSK3β,⁷ has a remarkable ability to prevent and/or reverse the full ACM disease phenotype (arrhythmias, exercise-induced sudden death, ventricular myocyte injury and apoptosis, inflammation, and contractile dysfunction) in multiple in vitro and in vivo models of ACM, and in human iPSC-cardiac myocytes derived from ACM patients.^{6,7} See also US2017/0097363. Thus the clinically important features of the disease phenotype—arrhythmias and myocardial damage—arise via a common disease mechanism that can be blocked by a single small molecule (SB216763).

GSK3β acts on and with a large and varied number of other signaling molecules; in inflammation crosstalk between pathways complicates the picture even further. See, e.g., Hoesel and Schmid, Mol Cancer. 2013; 12: 86. As shown herein, ACM disease alleles activate NFκB signaling in cardiac myocytes. Surprisingly, inhibition of this signaling system is as effective as SB216763 in preventing the full ACM disease phenotype. This provides new evidence that ACM is an inflammatory disease and that NFκB-targeted anti-inflammatory therapy is a powerful, mechanism-based approach to reduce adverse events in ACM patients.

One of the more striking observations in this study is the production and secretion of diverse pro-inflammatory cytokines and chemoattractants by ACM patient-derived cardiac myocytes grown under basal conditions in vitro. In previous studies of such cell lines,⁴⁹ it was necessary to use a combination of provocative stimuli (dexamethasone, 3-isobutyl-1-methylxanthine, rosiglitazone and indomethacin) to induce metabolic changes seen in patients with ACM. By contrast, we showed that expression of a common variant in PKP2 is sufficient to induce marked expression of immune mediators by human cardiac myocytes under basal conditions and in the absence of inflammatory cells. This observation, combined with results from in vitro and in vivo experimental models (which involved 2 different desmosomal mutations) suggests that activation of an innate immune response in cardiac myocytes occurs as a cell autonomous process in response to multiple ACM disease alleles independent of the actions of professional inflammatory cells.

Although our results to do not prove that cytokines are responsible for causing myocardial damage and arrhythmias in ACM, there was a clear correlation between activation of an immune response and expression of the disease phenotype.

Expression levels of two cytokines in particular, LIX (CXCL5) and osteopontin (OPN), were found to correlate with ejection fraction in Dsg2^{mut/mut} mice. LIX was increased by >50-fold in Dsg2^{mut/mut} mice and its level was markedly reduced in ACM mice treated with Bay 11-7082. Production of LIX is stimulated by IL-1(3 and TNFα. It promotes chemotaxis of neutrophils and also plays a role in fibrosis. OPN expression was increased by >40-fold in Dsg2^{mut/mut} mice and it too was reduced by Bay 11-7082. OPN regulates cell adhesion and survival. It also acts as a Th1 cytokine and participates in cell-mediated immune responses. In turn, expression of LIX and OPN was correlated with expression of other mediators including CCL21 (a T-cell and dendritic cell attractant), complement factor D (required for activation of the alternative pathway), DPP-IV (a dipeptidyl peptidase involved in immune regulation and apoptosis), GAS6 (which plays a role in fibrosis), IFNγ, IL-1Ra and IL-27 (which induces T-cell differentiation and upregulates IL-10 which itself was increased in ACM mice). These observations suggest that networks of immune mediators, likely derived from both cardiac myocytes and infiltrating inflammatory cells, interact in a complex fashion to promote the ACM disease phenotype.

Our results raise the possibility that targeting immune signaling could be an effective mechanism-based therapy in ACM. This notion is in keeping with recent insights into the role of immune activation in coronary artery disease and heart failure. Numerous drugs that block NFκB signaling are approved by the US Food and Drug Administration, mainly for treating cancer.⁵⁴ In the CANTOS trial, a monoclonal antibody against IL-1β significantly reduced major adverse cardiac events in patients with coronary artery disease.³⁶ The fact that IL-1β expression was increased by ˜13-fold in in Dsg2^{mut/mut} mice warrants further investigation as a possible therapeutic strategy in ACM. Finally, strenuous exercise is known to accelerate disease penetrance and increase arrhythmic risk in ACM patients.^{4,5} It remains to be determined if exercise intensifies the immune response in ACM and, if so, whether anti-inflammatory therapy might mitigate its adverse effects.

Methods of Treatment

In some embodiments, the methods described herein include administering a treatment comprising an inhibitor of NFκB to a subject identified as having ACM or being at risk for ACM (i.e., based on family history or the presence of genetic mutations associated with ACM). A subject can be identified as having ACM (diagnosed with ACM) based on methods known in the art, and/or using the methods described in US2017/0097363. A diagnosis usually rests on fulfilling a set of clinical criteria; see, e.g., Marcus et al., Circulation, 2010; 121:1533-1541.

The subject to be treated with the present methods can be any mammal, e.g., a human or non-human mammal (e.g., a veterinary or zoological subject). In preferred embodiments, the subject is a human.

The methods can also include recommending or advising the subject to avoid strenuous or intense physical activity or exercise; recommending or prescribing or administering one or more Singh Vaughan Williams class II antiarryhthmics (beta blockers) such as propranolol, esmolol, timolol, metoprolol, or atenolol; recommending or prescribing or administering one or more class III anti-arrhythmics (K-channel blockers) such as amiodarone, sotalol, ibutilide, dofetilide, dronedarone or E-4031; recommending or performing cardiac ablation; or recommending or implanting an implantable cardiac defibrillator (ICD).

Without wishing to be bound by theory, it is believed that desmosomal mutations activate GSK3β, which stimulates NFκB signaling in cardiac myocytes and promotes myocardial inflammatory cell infiltration. Cytokines produced by cardiac myocytes and/or infiltrating inflammatory cells may act by autocrine and/or paracrine actions to further stimulate NFκB signaling in cardiac myocytes. In most settings, NFκB signaling is turned on by a specific stimulus, such as an invading pathogen, but once the offending agent has been eliminated, the pathway turns off. In ACM, however, the stimulus (GSK3β) is persistent and it is intensified by exercise. Therefore, effective drug therapy in ACM will likely require chronic administration. This idea is supported by previous unpublished observations in which arrhythmias ceased within 24 hours in ACM mice treated with SB216763, but returned within 48 hours of cessation of treatment. Meanwhile, treatment with NFκB inhibitors is advantageous in that the potential adverse effects caused by long-term use of Wnt agonists (e.g. GSK3β blocker) is alleviated or eliminated. Thus, the methods can include administration of an inhibitor of NFκB once or twice daily, every other day, every third day, twice a week, or using a sustained release formulation that provides an effective amount of the drug for one or more days, with the duration of administration being at least one week, two weeks, three weeks, one month, two months, three months, four months, five months, six months, nine months, one year, two years, three years, or longer, e.g., for the lifetime of the subject.

NFκB Inhibitors

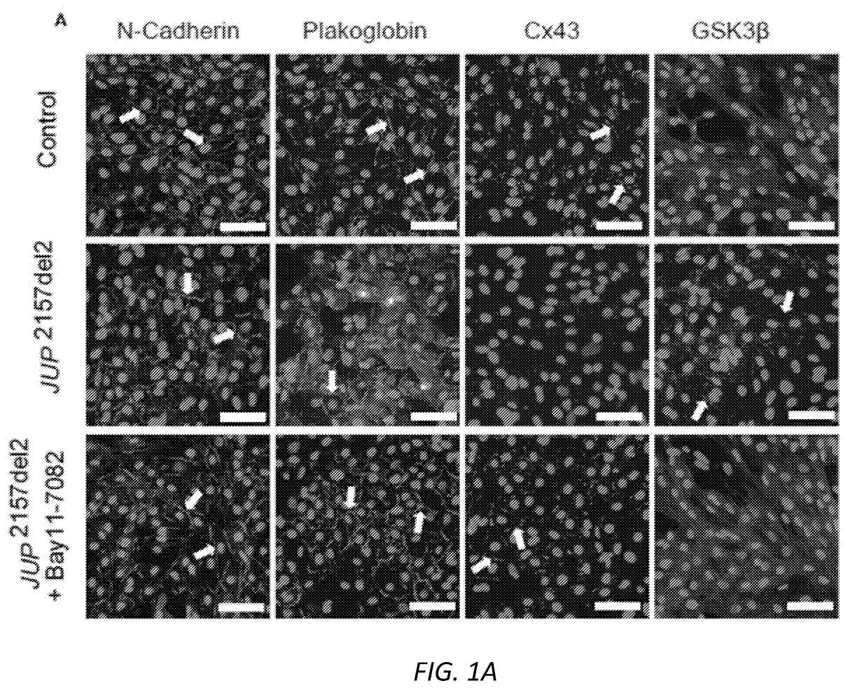

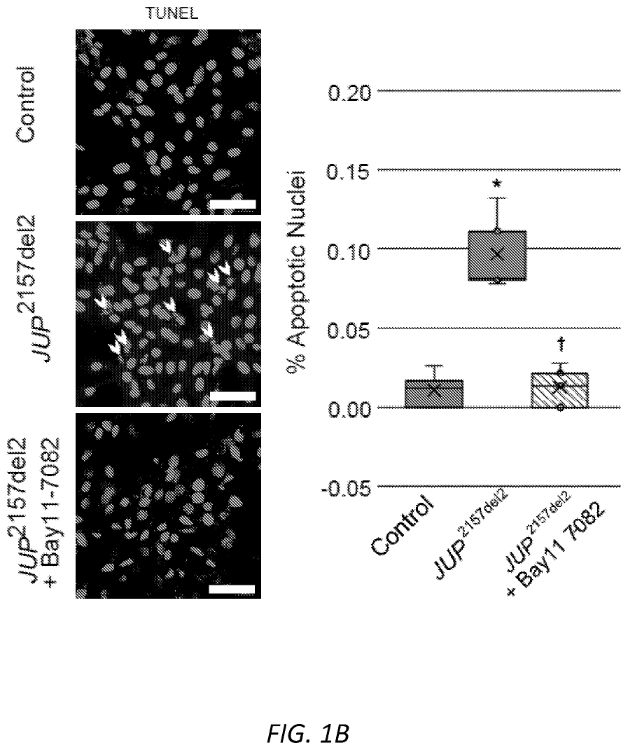

As simplified and shown in FIG. 1, stimulation of cell surface receptors by cytokines, LPS, antigens, etc., activates an IKK (IκB kinase) complex that phosphorylates IκB proteins, and thereby targets them for ubiquitination and degradation. The resulting free NFκB/Rel complexes are further activated by various post-translational modifications (phosphorylation, acetylation and/or glycosylation, reflecting actions of many regulatory enzymes), and translocate to the nucleus where they combine with other transcription factors to regulate gene expression in the immune response.

Inhibitors of NFκB thus could include the following types (from downstream to upstream): DNA binding inhibitors including GYY 4137, p-XSC, CV 3988, and Prostaglandin E2 (PGE2) that inhibit the binding between NFκB/Rel and its target DNA, thus inhibiting any gene expression activated by NFκB; inhibitors of post-translational modifications on NFκB/Rel, e.g. a p65 acetylation inhibitor, including Gallic acid and Anacardic acid that prevents NFκB from activating its target genes;

translocation inhibitors including JSH-23, and Rolipram that prevents NFκB/Rel from translocating to the nucleus; IκB degradation inhibitors including BAY 11-7082, MG-115, MG-132, Lactacystin, Epoxomicin, Parthenolide, Carfilzomib, and MLN-4924 (Pevonedistat) that prevents ubiquitinated IκB from being degraded, thus maintaining IκB's suppression of NFκB/Rel functions; IKK inhibitors including TPCA 1, NF-κB Activation Inhibitor VI (BOT-64), BMS 345541, Amlexanox, SC-514 (GK 01140), IMD 0354, and IKK-16 that prevent the phosphorylation of IκB and thus preventing the ubiquitination and degradation of IKB. In some embodiments, NFκB inhibitors are proteasome inhibitors including MG132, bortezomib, carfilzomib, and ixazomib. In some embodiments, NFκB inhibitors inhibit nuclear translocation inhibitors including dehydroxymethylepoxyquinomicin (DHMEQ), small peptidomimetics, such as SN-50, which encompasses the NLS of p50. In some embodiments, NFκB inhibitors inhibit NFκB's DNA binding, including sesquiterpene lactone (SL) compounds and decoy oligodeoxynucleotides.

Person of skills in the art will readily recognize additional types of NFκB inhibitors based on the mechanistic pathways involved, including, e.g., agents that can inhibit protein kinases, protein phosphatases, proteasomes, ubiquitnation, acetylation, methylation, and DNA binding steps have been identified as NF-κB inhibitors. (Pires et al., Genes (Basel). 2018 Janury 9; 9(1). pii: E24; Gupta, 2010 October-December; 1799(10-12):775-87).

The contents of Pires et al., Genes (Basel). 2018 Janury 9; 9(1) and Gupta et al., Biochim Biophys Acta., 2010 October-December; 1799(10-12) are hereby incorporated by reference. Table 1 of Gupta et al., Biochim Biophys Acta., 2010 October-December; 1799(10-12) lists some of the known NFκB inhibitors. Person of skills in the art will understand that the inhibitors may be small molecules, biologics, or other types of agents that block the function of NFκB. In some embodiments, the NFκB inhibitors are antibodies against targets affecting NFκB functions. The antibodies may be blocking antibodies or agonistic antibodies depending on the involvement of the antibody's target in NFκB functionality. Non-limiting examples of NFκB inhibitors also include 15d-PGJ(2), Calagualine, Conophylline, Evodiamine, Geldanamycin, Perrilyl alcohol, PSK, Rocaglamides, Adenovirus E1A, NSSA (Hep-C virus), Erbin overexpression, Golli BG21, KSR, MAST205, PEDF, Rituximab, TNAP, Betaine, Desloratadine, LY29 and LY30, MOL 294 , Pefabloc, Rhein, SMI and FP, [6]-gingerol, 1′-Acetoxychavicol acetate, 20(S)-Protopanaxatriol, 4-Hydroxynonenal, Acetyl-boswellic acids, Anandamide, Anethole, Apigenin, Artemisia vestital, Baoganning, Betulinic acid, Buddlejasaponin IV, Cacospongionolide B, Calagualine, Cardamomin, Casparol, Cobrotoxin, Cycloepoxydon, Decursin, Dehydroascorbic acid, Dexanabinol, Digitoxin, Diosgenin, Diterpenes, Docosahexaenoic acid, Falcarindol, Flavopiridol, Furonaphthoquinone, Garcinone B, Glycine chloramine, Guggulsterone, Herbimycin A, Honokiol, Hypoestoxide, Indirubin-3′-oxime, Isorhapontigenin, Clarithromycin, Cloricromene, C-K and Rh(2), Cryptotanshinone, Cytochalasin D, Danshenshu, Diterpenoids, Ent-kaurane diterpenoids, Epinastine hydrochloride, Epoxyquinol A, Erythromycin, Evodiamine, Fucoidan, Gallic acid, Ganoderma lucidum, Garcinol, Geranylgeraniol, Ginkgolide B, Glycyrrhizin, Halofuginone, Hematein, Herbal compound 861, Hydroxyethyl starch, Hydroxyethylpuerarin, Hypericin, Kamebakaurin, Linoleic acid, Lithospermi radix, Macrolide antibiotics, 2-methoxyestradiol, 6-MITC, Oridonin, Plant compound A, Polyozellin, Prenylbisabolane 3, Prostaglandin E2, PSK, Quinic acid, Sanggenon C, Sesamin, Shen-Fu, Silibinin, Sinomenine, Tansinones, Taurine+niacine, TZD MCC-555, Trichostatin A, Triptolide, Tyrphostin AG-126, Ursolic acid, Withaferin A, Xanthohumol, Xylitol, Yan-gan-wan, Yin-Chen-Hao, Ghrelin, Peptide YY, Rapamycin, Adiponectin, Kahweol, Manumycin A, Monochloramine, N-acetylcysteine, Nitric oxide, Nitrosylcobalamin, Oleandrin, Omega 3 fatty acids, ox-LDL, Panduratin A, PEITC, Petrosaspongiolide M, Phytic acid, Piceatannol, Pinosylvin, Plumbagin, Prostaglandin A1, Quercetin, Rengyolone, Rosmarinic acid, Rottlerin, Saikosaponin-d, Sanguinarine, Staurosporine, Sesquiterpene lactones, Scoparone, Silibinin, Silymarin, Sulforaphane, Sulindac, Tetrandine, Theaflavin, Thienopyridine, Tilianin, Ursolic acid, Vesnarinone, Wedelolactone, Withanolides, Xanthoangelol D, Zerumbone, β-carboline, γ-mangostin, γ-Tocotrienol, IKKβ peptide, NEMO CC2-LZ peptide, Anti-thrombin III, Chorionic gonadotropin, FHIT, HB-EGF, Hepatocyte growth factor, Interferon-α, Interleukin-10, PAN1, PTEN, SOCS1, Adenovirus, MC159, MC160, Angiopoietin-1, Antithrombin, β-catenin, Bromelain, CaMKK, CD43 overexpression, FLN29 overexpression, FLIP, G-120, Interleukin 4, Transdominant p50, VEGF, ADP ribosylation inhibitor, 7-amino-4-methylcoumarin, Amrinone, Atrovastat, Benfotiamine, Benzamide, Bisphenol A, Caprofen, Carbocisteine, Celecoxib, Germcitabine, Cinnamaldehyde, 2-methoxy CNA, 2-hydroxy CNA, CDS, CP Compound, Cyanoguanidine, HMP, α-difluoromethylornithine, DTD, Evans Blue, Evodiamine, Fenoldopam, FEX, Fibrates, FK778, Flunixin meglumine, Flurbiprofen, Hydroquinone, IMD-0354, JSH-21, KT-90, Lovastatin, Mercaptopyrazine, Mevinolin, Monoethylfumarate, Moxifloxacin, Nicorandil, Nilvadipine, NO-ASA, Panepoxydone, Peptide nucleic acids, Perindopril, PAD, α-PBN, Pioglitazone, Pirfenidone, PNO derivatives, Quinadril, AIDCA derivative, TDZD, TPCA-1, Pyridine derivatives, ACHP, Acrolein, AGRO100, Amino-pyrimidine, AS602868, Aspirin, Azidothymidine, BAY-11-7082, BAY-11-7083, Benzoimidazole derivative, Benzyl isothiocyanate, BMS-345541, Carboplatin, CDDO-Me, CHS 828, Compound 5, Compound A, Cyclopentenones, CYL-19s, CYL-26z, Diaylpyridine derivative, DPE, Epoxyquinone, Gabexate mesilate, Gleevec, Hydroquinone, Ibuprofen, IQCAD, Indolecarboxamide, Isobutyl nitrite, Jesterone dimer, 15-deoxyspergualine analog, Methotrexate, MLB120, Monochloramine, MX781 (Retinoid antagonist), 4-HPR, Nafamostat mesilate, NSAIDs, PS-1145 (MLN1145), PQD, Pyridooxazinone derivative, SC-514, Scytonemin, Sodium salicylate, Statins (several), Sulfasalazine, Sulfasalazine analogs, Survanta, Thalidomide, THI 52, YC-1, Lead, Mild hypothermia, Saline (low Na+), 5′-methylthioadenosine, Alachlor, Amentoflavone, Antrodia camphorata, Aucubin, Baicalein, Raxofelast, Ribavirin, Rifamides, Ritonavir, Rosiglitazone, Roxithromycin, DAAS, Serotonin derivative, Simvastatin, SM-7368, T-614, Sulfasalazine, SUN C8079, Triclosan plus CPC, Tobacoo smoke, Verapamil, Heat (fever-like), Hypercapnic acidosis, Hyperosmolarity, Hypothermia, Alcohol, 4′-DM-6-Mptox, 4-phenylcoumarins, AHUP, Luteolin, Mesuol, Nobiletin, Phomol, Psychosine, Qingkailing, Saucerneol D & E, Shuanghuanglian, Trilinolein, Wortmannin, α-zearalenol, NF-kappaB-repression factor, PIAS3, PTX-B, 17-AAG, TMFC, AQC derivatives, 9-aminoacridine derivatives, Chromene derivatives, D609, Dimethylfumarate, EMDPC, Histidine, Mesalamine, PEITC, Pranlukast, RO31-8220 (PKC, inhibitor), SB203580 (MAPK inhibitor), Tetrathiomolybdate, Tranilast,

Troglitazone, Catalposide, Cyclolinteinone, Dihydroarteanniun, Docosahexaenoic acid, Emodin, Ephedrae herba (Mao) extract, Equol, Erbstatin, Ethacrynic acid, Fosfomycin, Genipin, Genistein, Glabridin, Glucosamine sulfate, Isomallotochromanol, Isomallotochromene, Melatonin, Midazolam, Momordin I, Polymyxin B, Prostaglandin, Resiniferatoxin, Thiopental, Tipifarnib, TNP-470, Ursodeoxycholic acid, β-PEITC, 8-MSO, β-lapachone, Penetratin, VIP, Activated protein C, HSP-70, Interleukin-13, Intravenous Ig, Murrl gene product, Neurofibromatosis-2 protein, PACAP, SAIF, α-MSH, γ-glutamylcysteine synthetase, 1-Bromopropane, Acetaminophen, Diamide, Dobutamine, Cyclosporin A, Lactacystine, β-lactone, APNE, Boronic acid peptide, BTEE, 3,4-dichloroisocoumarin, Deoxyspergualin, DFP, Disulfiram, FK506 (Tacrolimus), Bortezomib, Salinosporamide A, 23-hydroxyursolic acid, Anetholdithiolthione, Apocynin, Arctigenin, Aretemisa p7F, Astaxanthin, Benidipine, bis-eugenol, BG compounds, BHA, CAPE, Carnosol, Carvedilol, Catechol derivatives, Celasterol, Cepharanthine, Chlorogenic acid, Chlorophyllin, Curcumin, DHEA, DHEA sulfate, Dehydroevodiamine, Demethyltraxillagenin, Diethyldithiocarbamate, Diferoxamine, Dihydroisoeugenol, Dihydrolipoic acid, Dilazep, Fenofibric acid, DMDTC, Dimethylsulfoxide, Disulfiram, Ebselen, Edaravone, EGTA, EPC-K1, Epigallocatechin-3-gallate, Ergothioneine, Ethyl pyruvate, Garcinol, γ-glutamylcysteine synthetase, Glutathione, Hematein, Hydroquinone, Hydroquinone, IRFI 042, Iron tetrakis, Isovitexin, Kangen-karyu extract, Ketamine, Lacidipine, Lazaroids, L-cysteine, Lupeol, Magnolol, Maltol, E-73, Ecabet sodium, Gabexate mesilate, Glimepiride, Hypochlorite, Losartin, LY294002, Pervanadate, Phenylarsine oxide, Phenytoin, Ro106-9920, Sabaeksan, U0126 (MEK inhibitor), 15-deoxyspergualin, 2′,8″-biapigenin, 5F (from Pteri syeminpinnata), Alginic acid, Apigenin, Astragaloside IV, AT514 (serratamolide), Atorvastatin, Cantharidin, Chiisanoside, Clarithromycin, Eriocalyxin B, Hirsutenone, JM34, KIOM-79, Leptomycin B, Neomycin, Nucling, Oregonin, OXPAPC, Paeoniflorin, Phallacidin, Piperine, Pitavastatin, Rapamycin, Selenomethionine, Shenfu, Sopoongsan, Sphondin, T. polyglycosides, Younggaechulgam-tang, a-pinene, NCPP, PN50, Mangiferin, Melatonin, Mn-SOD, Myricetin, N-acetyl-L-cysteine, Nacyselyn, Naringin, N-ethyl-maleimide, Nitrosoglutathione, NDGA, Ochnaflavone, Orthophenanthroline, Phenylarsine oxide, Pyrithione, Pyrrolinedithiocarbamate, Quercetin, Quinozolines, Rebamipide, Redox factor 1, Resveratrol, Rotenone, Roxithromycin, S-allyl-cysteine, Sauchinone, Sodium 4-Aminosalicylate, Spironolactone, Taxifolin, Tempol, Tepoxaline, tert-butyl hydroquinone, Tetracylic A, Wogonin, xanthohumol, Yakuchinone A, B, α-lipoic acid, α-tocopherol, α-torphryl acetate, α-torphryl succinate, β-Carotene, Diltiazem, Dioxin, Dipyridamole, Disulfiram, Enalapril, Fluvastatin, Indole-3-carbinol, JSH-23, KL-1156, Leflunomide, Levamisole, Moxifloxacin, Omapatrilat, R-etodolac, Rolipram, SC236 (COX-2 inhibitor), Triflusal, Actinodaphine, Artemisinin, Baicalein, β-lapachone, Calcitriol, Campthothecin, Capsiate, and Catalposide. See, e.g., Gupta et al., Biochim Biophys Acta., 2010 October-December; 1799(10-12). In some embodiments, the NFκB inhibitor is Andrographolide; Bay 11-7082; Bithionol; Bortezomib; CBL0137 (CBL-0137); Cantharidin; Chromomycin A3; Daunorubicinum; Diethylmaleate; Digitoxin; Ectinascidin 743; Emetine; Evodiamine (Isoevodiamine); Fluorosalan; GSK2982772; GSK583; Indole-3-carbinol; JSH-23; Magnolol; Manidipine hydrochloride; Narasin; Lestaurtinib; Omaveloxolone (RTA-408); Ouabain; QNZ (EVP4593); (−)-Parthenolide; Pyrrolidinedithiocarbamate ammonium; Rapamycin; SC75741; Sorafenib tosylate; Sunitinib malate; Tioconazole; Tribromsalan; Triclabendazolum; Triptolide (PG490); or Zafirlukast. In some embodiments, the inhibitor is emetine, fluorosalan, sunitinib malate, bithionol, narasin, tribromsalan, lestaurtinib, ectinascidin 743, chromomycin A3, or bortezomib. See, e.g., Miller et al., Biochem Pharmacol. 2010 May 1; 79(9): 1272-1280. In some embodiments, the NFκB inhibitor is not sodium salicylate.

In some embodiments, the NFκB inhibitor is an inhibitor of IKK. IKK comprises two subunits IKKα and IKKβ. Each subunit is important for the phosphorylation of IκB. (Mazhar Adli, IKKα and IKKβ Each Function to Regulate NF-κB Activation in the TNF-Induced/Canonical Pathway, PLoS One. 2010; 5(2): e9428). In some embodiments, an additional component of IKK is IKKγ/NEMO. In some embodiments, the NFκB inhibitor inhibits the function of IKKα; in some embodiments, the NFκB inhibitor inhibits the function of IKKβ; and in some embodiments, the NFκB inhibitor inhibits both the function of IKKα and the function of IKKβ. In some embodiments, the NFκB inhibitor inhibits IKKγ/NEMO. IKK inhibitors can include ATP analogs, allosteric modulators, and agents interfering with the kinase activation loops. (Begalli et. al., Unlocking the NF-KB Conundrum: Embracing Complexity to Achieve Specificity, Biomedicines. 2017 Aug. 22; 5(3). pii: E50). Examples of ATP analogues include β-carboline, SPC-839, BMS-345541, and SAR-113945. In some embodiments, the NFκB inhibitor is Bay 11-7082. Bay 11-7082 inhibits both IKKa and the function of IKKβ. (Rauert-Wunderlich, The IKK inhibitor Bay 11-7082 induces cell death independent from inhibition of activation of NFκB transcription factors, PLoS One. 2013; 8(3):e59292). Other known IKK inhibitors include IMD-0354 (N-(3,5-Bis-trifluoromethylphenyl)-5-chloro-2-hydroxybenzamide), TPCA 1, NF-κB Activation Inhibitor VI (BOT-64), BMS 345541, Amlexanox, SC-514 (GK 01140), IMD 0354, and IKK-16. The contents of Begalli F et. al., Unlocking the NF-κB Conundrum: Embracing Complexity to Achieve Specificity, Biomedicines. 2017 Aug. 22; 5(3). pii: E50, and Rauert-Wunderlich et. al., The IKK inhibitor Bay 11-7082 induces cell death independent from inhibition of activation of NF-κB transcription factors, PLoS One. 2013; 8(3):e59292 are hereby incorporated by reference.

Various NFκB inhibitors are readily available through public sources. For example, Santa Cruz Biotechnology provides NFκB inhibitors for purchases, including BAY 11-7085, Helenalin, NFkappaB Activation Inhibitor II, JSH-23, QNZ (EVP4593), Andrographolide, etc. (Santa Cruz Biotechnology). Various anti-NFκB antibodies, for example, are available for purchase at Sigma-Aldrich, as well as antibodies against other proteins involved in NFκB functionality, e.g., anti-IKK antibodies available at Sigma-Aldrich.

### EXAMPLES The invention is further described in the following examples, which do not limit the scope of the invention described in the claims.

### Materials and Methods

The following materials and methods were used in the examples set forth herein.

### Animal Studies

All animal studies were in full compliance with policies of Beth Israel Deaconess Medical Center, Johns Hopkins School of Medicine and St. George's University of London. They also conformed to the Guide for the Care and Use of Laboratory Animals from the National Institutes of Health (NIH publication no. 85-23, revised 1996). In vitro studies involved transfection of cultured neonatal rat ventricular myocytes with a mutant allele of the desmosomal protein plakoglobin (2157de12) as previously described.¹⁵ In vivo studies were performed in mice with homozygous knock-in of a mutant form of Dsg2, the gene encoding the desmosomal cadherin desmoglein-2 (Dsg2^{mut/mut} mice) as previously described.⁶ This mutation entails loss of exons 4 and 5 which causes a frameshift and premature termination of translation.

### Preparation of Primary Cultures of Neonatal Rat Ventricular Myocytes

Primary cultures were prepared from disaggregated ventricles of 1-day-old Wistar rat pups (Charles River) as previously described.¹⁵ Cell suspensions were pre-plated to reduce fibroblast content, placed in collagen-coated chamber slides at a density of 2.4×10⁵ cells/cm², and grown for 4 days at 37° C. in M199 medium (GIBCO) (supplemented with penicillin, 20 U/mL; streptomycin, 20 μg/mL; 10% neonatal calf serum and 0.1 mM bromodeoxyuridine) in a humidified atmosphere containing 1% CO₂. Epinephrine (0.01 μmol/mL) was added during the first 24 hours of culture. At 24 hours post-seeding, cultures were transfected with an adenoviral construct expressing plakoglobin (JUP) with the 2157del2 mutation, as previously reported.¹⁵ At 24 hours post-transfection, cultures were treated with Bay11-7082 (Sigma, 5 mM) for an additional 24 hours. Transfected cultures treated with vehicle only (DMSO) and non-transfected cultures were used for control purposes.

### In Vitro Immunofluorescence

Primary cultures of rat ventricular myocytes were washed in phosphate buffered saline (PBS), fixed with 4% paraformaldehyde in PBS, and permeabelized with 0.2% Triton X-100 in PBS. Cells were blocked with PBS containing 1% Triton X-100, 3% normal goat serum and 1% bovine serum albumin. Cells were then incubated first with primary antibodies and then with indocarbocyanine (Cy3)-conjugated goat anti-mouse IgG (Jackson Immunolabs, 1:400). Primary antibodies included mouse monoclonal anti-Cx43 (Millipore MAB3067, 1:200), anti-N-cadherin (SIGMA C1821, 1:400), anti-plakoglobin (SIGMA P8087, 1:800), and anti-GSK3β (Cell Signaling Technology, 27C10, 9315S, 1:100). DAPI was used to visualize nuclei. Immunostained preparations were visualized by confocal microscopy.

### In Vitro Apoptosis Assays

Terminal deoxynucleotidyl transferase dUTP nick end labeling (TUNEL) was performed on neonatal rat myocyte cultures according to the manufacturer's protocol (Millipore, S7110). The number of TUNEL-positive nuclei was counted in 5 randomly selected high-power fields in each culture well and expressed as a per cent of total nuclei visualized with DAPI.

### In Vitro Cytokine Assays

Culture media from cells expressing 2157del2 plakoglobin in the presence or absence of Bay11-7082 (5 μM, 24 hours) were collected after 96 hours in culture, mixed with a cocktail of biotinylated detection antibodies, and incubated with nitrocellulose membranes spotted in duplicate with control and capture antibodies (R&D Systems). Chemiluminescent signal produced at each spot corresponded to the amount of bound cytokine. Conditioned media from non-transfected cultures were assayed for cytokine expression as controls. The cytokine array specifically detected rat cytokines whereas cells were grown in media containing neonatal bovine serum. Thus, cross contamination was presumably insignificant.

### In Vivo Drug Treatment

Three cohorts of mice at 8 weeks of age were anesthetized and implanted with subcutaneous osmotic mini-pumps (Alzet, Model 1004). These included: 1) Dsg2^{mut/mut} mice with pumps containing 50 μg/μL Bay11-7082 in a vehicle of 65% DMSO, 15% ethanol, and 20% saline; 2) Dsg2^{mut/mut} mice with pumps containing an equivalent volume of vehicle; and 3) wildtype mice with pumps containing vehicle. Mice in cohort 1 received 5 mg/kg/day of Bay11-7082 by continuous infusion; those in cohorts 2 and 3 received an equivalent volume of vehicle each day for 4 weeks. At 12 weeks of age, mice were re-anesthetized and new Alzet pumps were implanted for an additional 4 weeks of treatment.

### Mouse Echocardiography and Electrocardiography

At the end of the 8 week drug treatment protocol, cardiac function was assessed in all mice by transthoracic echocardiography and electrocardiography (ECG) as previously described. Echocardio-graphic measurements were made according to American Society of Echocardiography guidelines⁴⁸ in non-sedated mice using a Vevo 2100 Visualsonic imaging system. Parasternal long-axis views of the left ventricle (LV), at the level of the papillary muscles, were acquired at a sweep speed of 200 mm/second. Three to five measurements were acquired from each mouse and averaged to assess LV function as previously described. ECG measurements made in anesthetized mice in which wire electrodes had been sutured in place in the right and left arms to obtain standard lead I ECG recordings. Recordings were captured via PowerLab and analyzed via the LabChart Pro ECG Analysis Add-on Software (LabChart Pro 8, MLS360/8, AD Intruments). ECGs were recorded for 15 minutes and all ECG wave and amplitude parameters were analyzed as signal averaged ECGs (SAECGs). Following completion of functional studies, mice were euthanized and hearts were excised and processed for additional studies either by freezing fresh tissue (−80C) or fixing tissue in 10% buffered formalin and embedding in paraffin.

### Mouse Myocardial Tissue Immunofluorescence and Immunoperoxidase Methods

Immunofluorescence staining was used to characterize the distribution of selected proteins at cell-cell junctions as previously reported.^{6,15} Deparaffinized slide-mounted myocardial sections (5 μm thick) were rehydrated and heated in citrate buffer (10 mmol/L, pH 6.0) to enhance specific immunostaining. After being cooled to room temperature, tissue sections were simultaneously permeabelized and blocked by incubating them in PBS containing 1% Triton X-100, 3% normal goat serum and 1% bovine serum albumin. The sections were then incubated first with primary antibodies and then with Cy3-conjugated goat anti-rabbit IgG (Jackson Immunolabs, 1:400). Primary antibodies included rabbit polyclonal anti-Cx43 (SIGMA C6219, 1:200), anti-N-cadherin (SIGMA C3678, 1:400), anti-plakoglobin (Thermo Fisher Scientific PA5-17320, 1:500), anti-GSK3β (Cell Signaling Technology 27C10, 9315S, 1:80) and anti-SAP97 (Abcam, ab134156, 1:200). Immunostained preparations were visualized by confocal microscopy. Additional sections prepared with Masson trichrome and hematoxylin & eosin stains were examined by light microscopy.

Immunoperoxidase staining was used to detect expression of selected cytokines in sections of mouse myocardium as previously reported.¹² Slide-mounted paraffin sections were heated to 60° C. for 80 minutes, cooled to room temperature, deparaffinized, and rehydrated. Antigen retrieval was achieved by heating the samples in citrate buffer (pH 6.0) to boiling followed by cooling to room temperature. Immunohistochemical staining was performed using conventional methods (Universal DAKO EnVision System; peroxidase). Once endogenous peroxidase activity had been blocked, primary antibody was applied followed by incubation with horseradish peroxidase-labeled polymer. The reaction was completed with an enzyme/substrate system, with diaminobenzidine as the chromogen substrate. The tissue was counterstained with Harris hematoxylin. Primary antibodies included rabbit polyclonal anti-TNFα (AbCam ab34674, 1:50 dilution), anti-IL-1(3 (AbCam ab9722, 1:500) and anti-MCP-1α (Novus Biologicals NBP2-41209, 1:250). In each experiment, control, vehicle-treated and drug-treated samples were batched to ensure identical staining and signal-generating conditions.

Immunoperoxidase staining using conventional techniques was also used to detect T-cells and macrophages in myocardial tissue sections from Dsg2^{mut/mut} mice. T-cells were identified using an anti-CD3 antibody (Abcam ab16669, 1:200 dilution) and macrophages were marked with an anti-CD68 antibody (Abcam ab127055, 1:400). After standard development in diaminobenzidine, sections were counterstained with hematoxylin and examined qualitatively by light microscopy.

hiPSC-Cardiac Myocyte Differentiation and Bay11-7082 Treatment

These studies involved a previously characterized hiPSC line from an ACM patient with a c.2013delC (p.672fsX683) mutation in the PKP2 gene originally produced by Joseph Wu at Stanford and described previously,^{7,49} and an unrelated healthy control hiPSC line derived at Johns Hopkins by Gordon Tomaselli and colleagues. Stem cell lines were cultured as monolayers and differentiated into cardiac myocytes according to a previously published protocol with minor modifications.⁵⁰ Briefly, hiPSCs were plated in 6-well culture plates coated with 1:200 Geltrex LDEV-free reduced growth factor matrix:DMEM/F-12 with HEPES (both Gibco). They were maintained for the first 18 hours in Essential 8 medium (E8, Gibco) with 10 μM Y-27632 dihydrochloride (Tocris Bioscience, Bristol, UK), and then subsequently fed daily with E8 medium for a total of four days. Cells were plated at a density sufficient to produce 70-90% confluence at 4 days, at which time differentiation was initiated (defined as day 0 or “d0”).

On d0, E8 media was replaced with RPMI 1640 medium supplemented with B-27 supplement minus insulin (Gibco) and 6 μM CHIR-99021 (Selleck Chemicals, Houston, Tex.) to initiate differentiation. Thereafter, media was changed as follows: RPMI 1640 medium with B-27 supplement (minus insulin) on d2, RPMI 1640 medium with B-27 supplement (minus insulin) and 5 μM IWR-1 (Sigma-Aldrich Corp., St. Louis, Mo.) on d3, RPMI 1640 medium with B-27 supplement (minus insulin) on d5 and d7, and RPMI 1640 medium with B-27 supplement (with insulin) on d9 and beyond. Spontaneous beating was first observed at d7-9. At d13-15, cells were dissociated using 0.05% trypsin-EDTA, mixed and re-plated on fresh Geltrex-coated 6 well plates. They were then metabolically-purified using lactate supplemented glucose-free DMEM/F12 for 4 days, with media changed every other day. Cells were then switched back to B-27 supplemented-RPMI and either maintained for further use or cryopreserved.

On day 30 from the start of differentiation, myocytes were plated at a density of 300,000 cells/cm² to produce confluent monolayers in 12-well plates or on thermonox coverslips in 24-well plates, both coated with 1:200 Geltrex:DMEM/F12. They were cultured in B-27 supplemented-RPMI for 1 week and allowed to form a contracting syncytium. Half of the wells were treated with Bay 11-7082 (10 μM) for 8 days, with media replaced every other day. Media removed at each change was stored at −80 C for subsequent cytokine assays. At day 8, cells were lysed with TRIzol Reagent (Ambion, Life Technologies) and the lysate was stored at −80 C for cytokine assays.

Cytokine Assays in Mouse Hearts and Patient hiPSC-Cardiac Myocytes

Myocardium from mice was lyzed in RIPA buffer and cardiac myocytes from patient hiPSCs were lysed in TRIzol, and protein in lysates was quantified by standard BCA protein assay. Mouse (R&D Systems, Cat. No. ARY028) and Human (R&D Systems, Cat. No. ARY022B) Proteome Profiler XL Cytokine Arrays were blocked at room temperature for 1 hour before being incubated overnight at 4° C. with 200 μg of myocardial protein in blocking buffer supplied by manufacturer. Additional arrays were incubated overnight at 4° C. with 200 μl of hiPSC-cardiac myocyte culture medium according to the manufacturer's protocol for cell supernatants. Following overnight incubation, arrays were washed four times (10 minutes/wash), incubated at room temperature for 1 hour with Antibody Detection Cocktail, washed an additional four times (10 minutes/wash), then incubated with 1:2,000 Streptavidin-HRP in blocking buffer for 30 minutes at room temperature. Arrays were then washed four times and chemiluminescence was performed using the Chemi Reagent Mix included in the assay kits.

### Statistical Analysis

Data are presented as mean±SEM. Specific n-values are inset within each figure legend or table. P<0.05 was deemed statistically significant. As appropriate, associations between continuous dependent variables were tested using Student's paired/unpaired t-tests (binary independent variables) or one-way ANOVA (two or more variables). In the in vivo studies involving Dsg2^{mut/mut} mice, correlations between (i) ejection fraction and the amounts of myocardial fibrosis and apoptosis, (ii) cytokine expression levels and ejection fraction, and (iii) the levels of each individual cytokine against all other cytokines were made using Pearson's r analysis. Changes in cytokine levels in WT vs. Dsg2^{mut/mut} and/or untreated vs. Bay 11-7082-treated Dsg2^{mut/mut} mice were analyzed by two-way ANOVA with Tukey's post-hoc testing. The levels of any cytokines showing a significant change were then correlated with ejection fraction in each animal using Pearson's r analysis. In addition, the levels of all cytokines (n=111) were analyzed in a Pearson's correlation matrix table to correlate the expression level of each individual cytokine with all other cytokines.

### Example 1

### Inhibition of NFκB Signaling Reverses ACM Disease Features In Vitro

Primary cultures of neonatal rat ventricular myocytes that express a mutant form of the desmosomal protein plakoglobin (2157de12), known to cause ACM in patients,⁵¹ exhibit several features in vitro that are also seen in the hearts of ACM patients.^{6,7,12,16,45} These include redistribution of plakoglobin (also known as γ-catenin) from cell-cell junctions into the cytosol and nucleus; loss of cell-surface immunoreactive signal for the major cardiac gap junction protein, Cx43; redistribution of GSK3β from the cytosol to the cell surface; and myocyte apoptosis. We have shown previously that all of these features are normalized when transfected cells are incubated with SB216763, which inhibits GSK3β.^{6,7} As shown in FIG. 1, these changes are also reversed when transfected cells are incubated with the NFκB blocker Bay 11-7082. The pattern of distribution of plakoglobin, Cx43 and GSK3β in transfected cells treated with Bay 11-7082 for 24 hours was indistinguishable from that seen in control (non-transfected) cardiac myocytes. Myocyte apoptosis, measured by TUNEL labeling, was increased by roughly 10-fold in ACM myocytes but returned to control levels after exposure to Bay 11-7082 for 24 hours (FIG. 1).

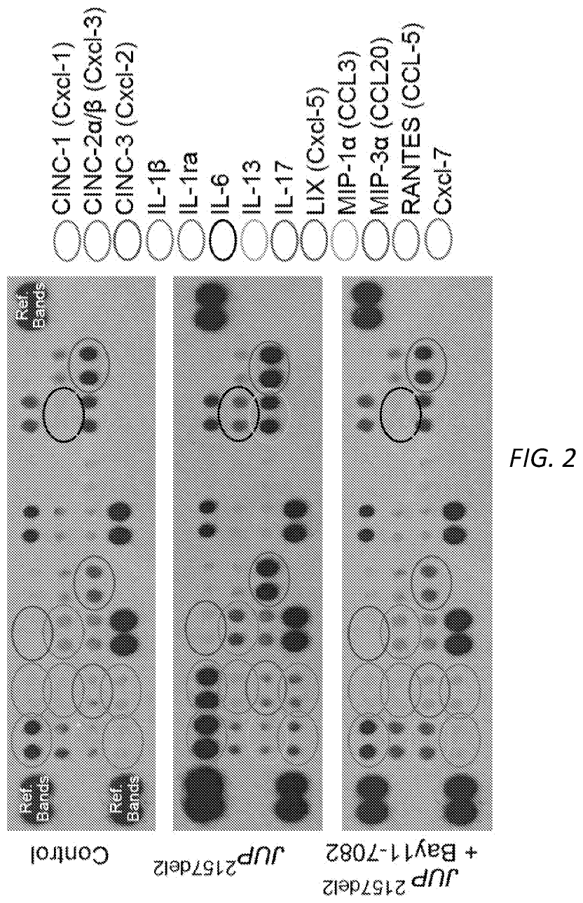

We have reported previously that expression of 2157del2 plakoglobin caused neonatal rat ventricular myocytes to produce and secrete cytokines into the culture media.⁷ A repeat of these studies confirms that cells that expressed mutant plakoglobin produced and secreted a wide variety of inflammatory cytokines and chemoattractant molecules (FIG. 2). In addition, we now show that incubating these cells with Bay 11-7082 for 24 hours greatly reduced accumulation of these factors in the culture media and produced a picture nearly identical to that seen in control (non-transfected) myocytes (FIG. 2). Because these cultures contain no leukocytes and consist of >90% cardiac myocytes, these observations indicated that 2157del2 plakoglobin stimulates secretion of inflammatory mediators by cardiac myocytes under the control of NFκB.

### Example 2

### Inhibition of NFκB Signaling Prevents Development of ACM Disease Features In Vivo

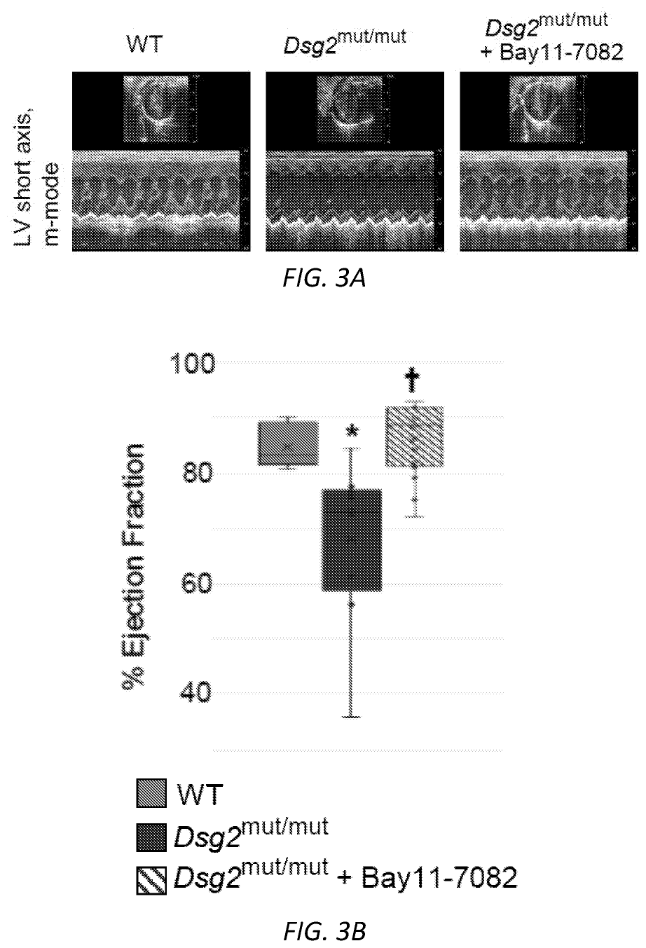

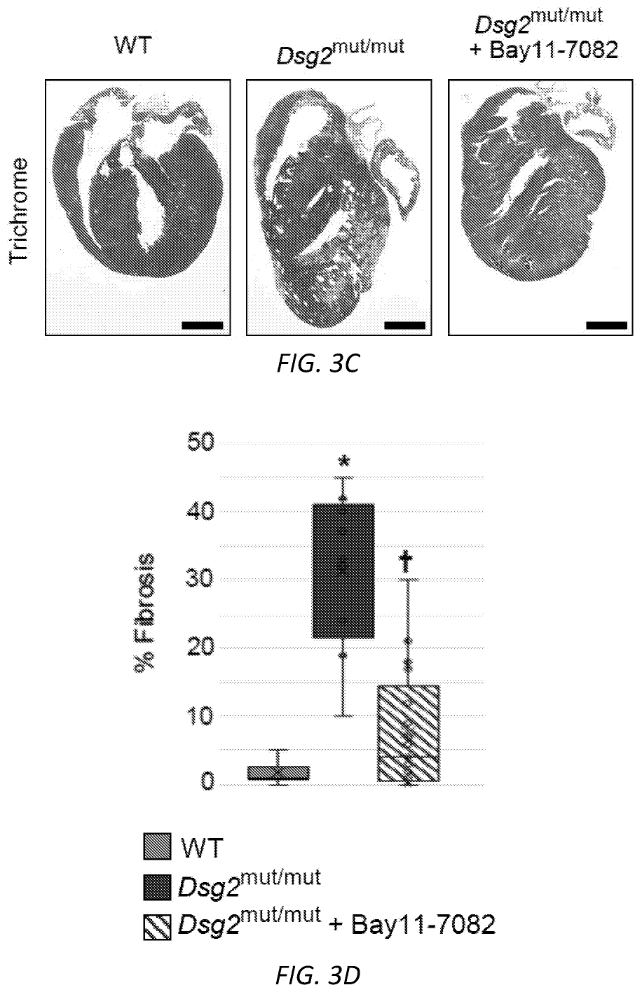

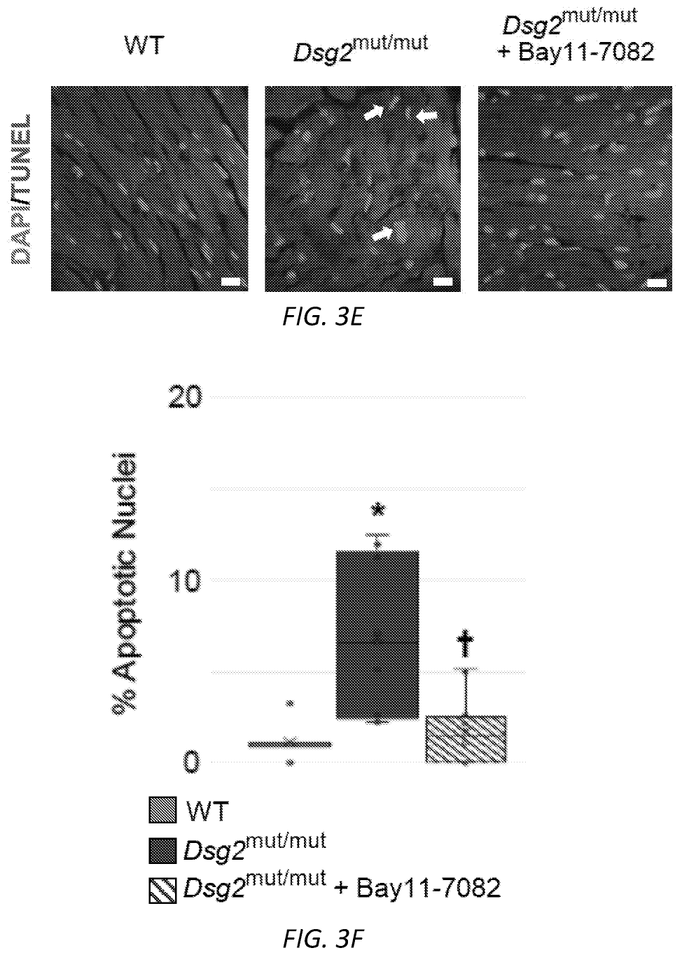

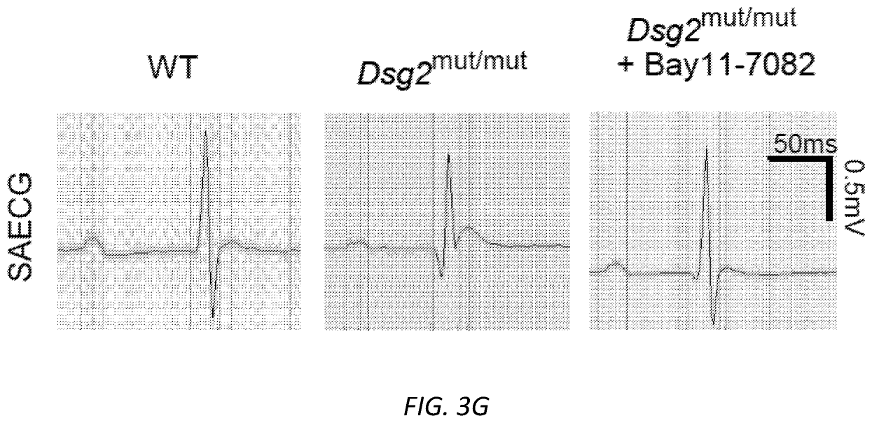

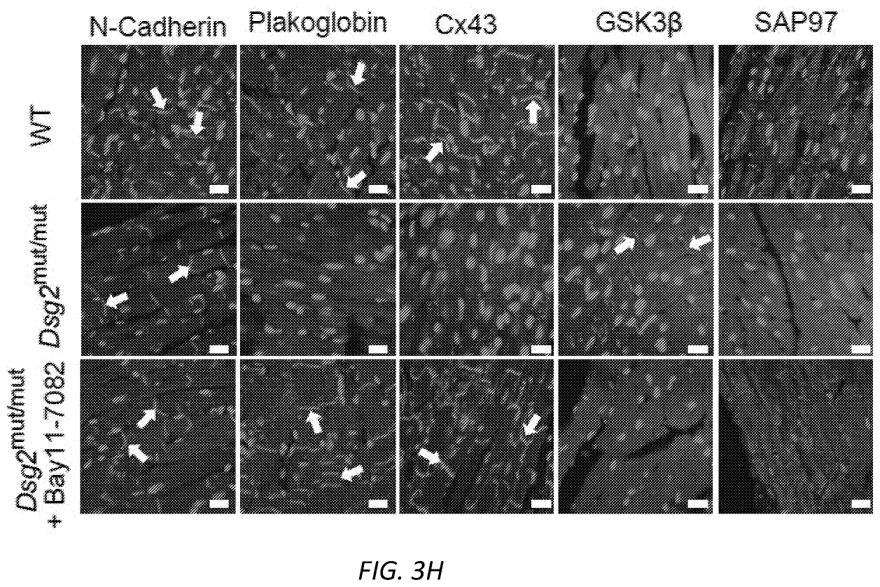

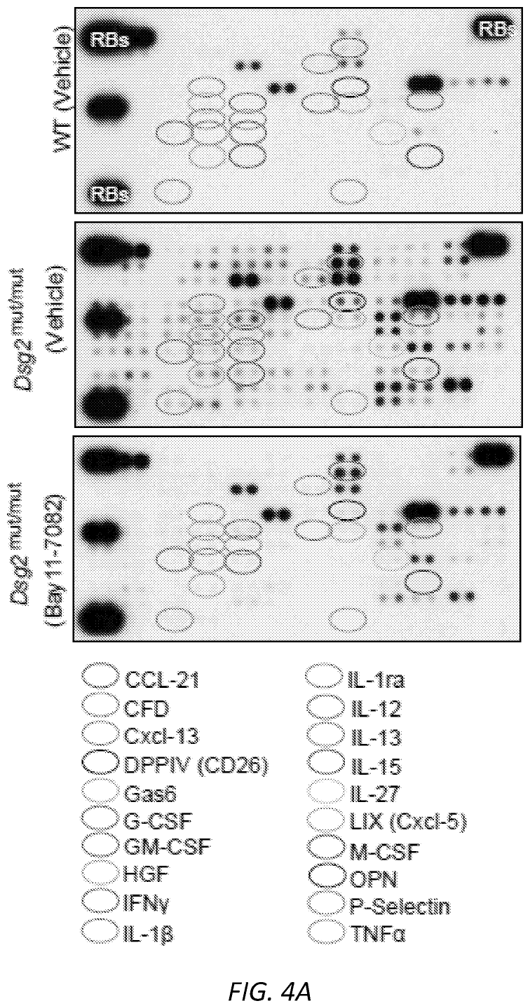

To determine if inhibition of inflammatory signaling mitigates development of the ACM phenotype in vivo, we treated Dsg2^{mut/mut} mice with Bay 11-7082 by continuous infusion over an 8 week period beginning when the mice were 8 weeks of age. As reported previously,⁶ DSg2^{mut/mut} mice showed little if any apparent cardiac structural or functional derangements at 8 weeks of age, but during the ensuing 8 weeks, they developed a robust phenotype that recapitulated the most important clinical features seen in ACM patients, namely myocardial damage and arrhythmias. This included progressive deterioration of ventricular contractile function associated with development of extensive myocardial necrosis, fibrosis and inflammation. It also included ECG abnormalities and arrhythmias. These structural and functional changes were associated with marked shifts in the distribution of various cardiac myocyte proteins including desmosomal proteins, connexins and ion channel proteins, proteins involved in the Wnt/β-catenin signaling pathway, and SAP97, a chaperone protein involved in ion channel transport to intercalated disks.^{6,7} As shown in FIG. 3, treatment of Dsg2^{mut/mut} mice with Bay 11-7082 substantially mitigated this phenotype. Contractile function was normalized and there was a marked reduction in the amount of ventricular myocardial necrosis and fibrosis, along with a significant reduction in the number of apoptotic cells seen by TUNEL labeling. Abnormalities in the signal averaged ECG were also corrected. Finally, abnormal distributions of plakoglobin, Cx43, GSK3β and SAP97, which also occur in patients with ACM,^{6,7,16} were fully corrected in Dsg2^{mut/mut} mice treated with Bay11-7082. Quantitative data for all morphologic, echocardiographic and electrocardiographic parameters measured in these mouse cohorts are shown in Table 1.

TABLE 1
Quantitative morphometric, echocardiographic
and electrocardiographic data

| | | | Dsg2^{mut/mut} + |
|---|---|---|---|
| Parameter | WT | Dsg2^{mut/mut} | Bay11-7082 |
| Morphometric | | | |
| n | 10 | 9 | 17 |
| RWT (mm) | 0.63 ± 0.02 | 0.55 ± 0.04^{†} | 0.64 ± 0.02 |
| LVM (mg) | 81.9 ± 4.9 | 96.3 ± 6.03* | 92.9 ± 3.08* |
| HW/BW (mg/g) | 4.6 ± 0.2 | 5.0 ± 0.2 | 4.7 ± 0.1 |
| Echocardiography | | | |
| n | 10 | 9 | 17 |
| FS (%) | 54.0 ± 1.5 | 32.9 ± 3.4*^{†} | 49.5 ± 1.9 |
| IVSd (mm) | 0.88 ± 0.03 | 0.91 ± 0.04 | 0.95 ± 0.02* |
| LVIDd (mm) | 2.87 ± 0.07 | 3.22 ± 0.17* | 2.96 ± 0.06 |
| LVIDs (mm) | 1.32 ± 0.05 | 2.16 ± 0.16*^{†} | 1.50 ± 0.08 |
| Electro- | | | |
| cardiography | | | |
| n | 10 | 9 | 17 |
| RR-I (ms) | 121 ± 2.8 | 119 ± 2.7 | 118 ± 2.5 |
| PR-I (ms) | 40.0 ± 0.9 | 40.3 ± 1.6 | 39.0 ± 0.9 |
| Pd (ms) | 11.7 ± 0.2 | 10.3 ± 0.6* | 10.1 ± 0.5* |
| QRSd (ms) | 12.5 ± 0.4 | 13.8 ± 1.5^{†} | 11.4 ± 0.4* |
| P-Amp (mV) | 0.08 ± 0.01 | 0.06 ± 0.01* | 0.06 ± 0.003* |
| R-Amp (mV) | 0.84 ± 0.07 | 0.55 ± 0.04*^{†} | 0.66 ± 0.04* |
| Q-Amp (mV) | −0.03 ± 0.01 | −0.11 ± 0.02*^{†} | −0.06 ± 0.01 |
| S-Amp (mV) | −0.25 ± 0.04 | −0.042 ± 0.05* | −0.09 ± 0.04* |

WT, wildtype; RWT, relative wall thickness; LVM, left ventricular mass; HW, heart weight; BS, body weight; FS, fractional shortening; IVSd, interventricular septum thickness at end-diastole; LVIDd, left ventricular internal diameter at end-diastole; LVIDs, left ventricular internal diameter at end-systole; RR-I, R-R internal; PR-I, P-R interval; Pd, P-wave duration; QRSd, QRS wave duration; P-Amp, P-wave amplitude; R-Amp, R-wave amplitude; Q-Amp, Q-wave amplitude; S-Amp, S-wave amplitude.
*P < 0.05 vs. WT;
^{†}P < 0.05 Dsg2^{mut/mut} vs. Dsg2^{mut/mut} + Bay11-7082

### Example 3

### Cytokines are Produced by Cardiac Myocytes and Infiltrating Inflammatory Cells in ACM

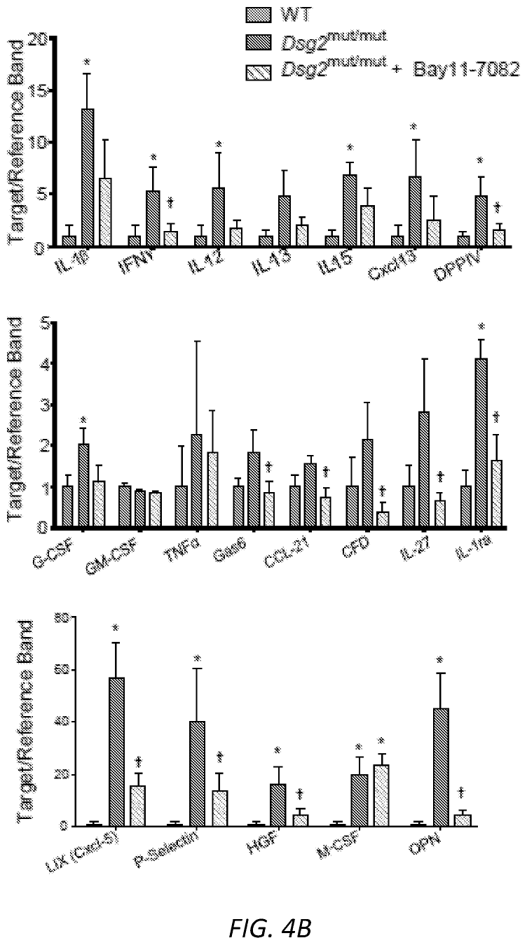

To characterize production of chemical mediators of the immune response in ACM, we used arrays to measure 111 different cytokines in the hearts of Dsg2^{mut/mut} mice and compared the amounts to those measured in the hearts of WT mice and Dsg2^{mut/mut} mice treated with Bay 11-7082. We observed substantial expression of multiple cytokines in the hearts of Dsg2^{mut/mut} mice. FIG. 4 shows data for selected cytokines (the complete data set is included in Table 2). Powerful inflammatory mediators were expressed in Dsg2^{mut/mut} hearts including IL-1β (up by ˜13-fold compared to WT hearts), IFNγ (˜5-fold), IL-12 (˜6-fold) and TNFα (˜2-fold). Similarly, various chemotactic molecules were greatly increased in Dsg2^{mut/mut} hearts compared to WT hearts, including the B-cell chemoattractant CXCL13 (up by ˜6-fold), M-CSF (˜20-fold), and the neutrophil chemoattractant LIX (CXCL5; ˜60-fold). And, expression of various pleomorphic molecules with multiple actions was also greatly increased including HGF (˜15-fold) and P-selectin (˜40-fold). Finally, there were increases in some molecules that fulfill anti-inflammatory roles such as IL-1Ra (up by ˜4-fold). In most, but not all, cases increased expression of these molecules in Dsg2^{mut/mut} hearts was blunted or fully normalized by treatment with Bay 11-7082 (FIG. 4).

TABLE 2
Cytokine expression levels for in vivo studies in mice.

| | | Dsg2^{mut/mut} | Dsg2^{mut/mut} |
|---|---|---|---|
| | Dsg2^{mut/mut} | (Bay11-7082) | (Bay11-7082) |
| Target | vs WT | vs WT | vs Dsg2^{mut/mut} |
| Adiponectin | <1 | <1 | <1 |
| Amphiregulin | <1 | <1 | <1 |
| Angiopoietin-1 | <1 | <1 | <1 |
| Angiopoietin-2 | <1 | <1 | 1-2 |
| Angiopoietin-like 3 | <1 | <1 | <1 |
| BAFF/BLyS/TNFSF13B | <1 | <1 | <1 |
| C1qR1/CD93 | 1-2 | <1 | <1 |
| CCL2/JE/MCP-1 | <1 | <1 | <1 |
| CCL3/CCL4/MIP-1a/B | <1 | <1 | <1 |
| CCL5/RANTES | <1 | <1 | <1 |
| CCL6/C10 | <1 | <1 | <1 |
| Eotaxin (CCL11) | <1 | <1 | <1 |
| MCP-5 (CCL12) | <1 | <1 | <1 |
| CCL17/TARC | <1 | <1 | <1 |
| CCL19/MIP-3B | <1 | <1 | <1 |
| CCL20/MIP-3a | 1-2 | <1 | <1 |
| CCL21/6Ckine | 1-2 | <1 | <1 |
| CCL22/MDC | <1 | <1 | <1 |
| CD14 | <1 | <1 | 1-2 |
| CD40/TNFRSF5 | <1 | <1 | <1 |
| CD160 | 1-2 | <1 | <1 |
| Chemerin | <1 | <1 | <1 |
| Chitinase 3-like 1 | <1 | <1 | <1 |
| Coagulation Factor III/ | <1 | <1 | <1 |
| Tissue Factor | | | |
| Complement Component | 1-2 | <1 | <1 |
| C5/C5a | | | |
| Complement Factor D | 2-4 | <1 | <1 |
| C-Reactive Protein/CRP | 1-2 | <1 | <1 |
| CX3CL1/Fractalkine | <1 | <1 | <1 |
| KC (CXCL1) | <1 | <1 | <1 |
| MIP-2 (CXCL2) | <1 | <1 | <1 |
| CXCL9/MIG | 1-2 | <1 | <1 |
| IP-10 (CXL10/CRG-2) | 1-2 | <1 | <1 |
| I-TAC (CXCL11) | 1-2 | <1 | <1 |
| CXCL13/BLC/BCA-1 | 6-10 | 2-4 | <1 |
| CXCL16 | 1-2 | <1 | <1 |
| Cystatin C | 1-2 | <1 | <1 |
| DKK-1 | 2-4 | <1 | <1 |
| DPPIV/CD26 | 4-6 | 1-2 | <1 |
| EGF | 2-4 | 1-2 | <1 |
| Endoglin/CD105 | <1 | <1 | <1 |
| Endostatin | 1-2 | <1 | <1 |
| Fetuin A/AHSG | 1-2 | <1 | <1 |
| FGF acidic | 1-2 | <1 | <1 |
| FGF-21 | 1-2 | <1 | <1 |
| FLt-3 Ligand | 2-4 | <1 | <1 |
| Gas 6 | 1-2 | <1 | <1 |
| G-CSF | 2-4 | 1-2 | <1 |
| GDF-15 | 1-2 | <1 | <1 |
| GM-CSF | <1 | <1 | <1 |
| HGF | >10 | 4-6 | <1 |
| sICAM-1 (CD54) | 1-2 | 1-2 | <1 |
| IFN-y | 4-6 | 1-2 | <1 |
| IGFBP-1 | 2-4 | 1-2 | <1 |
| IGFBP-2 | 2-4 | <1 | <1 |
| IGFBP-3 | 1-2 | <1 | <1 |
| IGFBP-5 | 1-2 | <1 | <1 |
| IGFBP-6 | 2-4 | <1 | <1 |
| IL-1a (IL-1F1) | 2-4 | <1 | <1 |
| IL-1β (IL-1F2) | >10 | 6-10 | <1 |
| IL-1ra (IL-1F3) | 4-6 | 1-2 | <1 |
| IL-2 | <1 | <1 | <1 |
| IL-3 | <1 | <1 | <1 |
| IL-4 | 1-2 | <1 | <1 |
| IL-5 | <1 | <1 | <1 |
| IL-6 | <1 | <1 | <1 |
| IL-7 | 1-2 | <1 | <1 |
| IL-10 | 1-2 | <1 | <1 |
| IL-11 | 1-2 | <1 | <1 |
| IL-12 p40 | 4-6 | 1-2 | <1 |
| IL-13 | 4-6 | 1-2 | <1 |
| IL-15 | 6-10 | 2-4 | <1 |
| IL-17a | <1 | <1 | <1 |
| IL-22 | <1 | <1 | <1 |
| IL-23 | <1 | <1 | <1 |
| IL-27 p28 | 2-4 | <1 | <1 |
| IL-28A/B | 1-2 | 1-2 | <1 |
| IL-33 | 2-4 | 2-4 | <1 |
| LDL R | <1 | 1-2 | 1-2 |
| Leptin | 2-4 | 1-2 | <1 |
| LIF | 1-2 | <1 | <1 |
| Lipocalin-2/NGAL | <1 | <1 | <1 |
| LIX | >20 | >10 | <1 |
| M-CSF | >10 | >20 | 1-2 |
| MMP-2 | 4-6 | 4-6 | 1-2 |
| MMP-3 | <1 | <1 | 1-2 |
| MMP-9 | <1 | <1 | <1 |
| Myeloperoxidase | 4-6 | 2-4 | <1 |
| Osteopontin (OPN) | >20 | 4-6 | <1 |
| Osteoprotegerin/ | <1 | <1 | <1 |
| TNFRSF11B | | | |
| PD-EGF/Thymidine | <1 | <1 | <1 |
| phosphorylase | | | |
| PDGF-BB | 2-4 | 1-2 | <1 |
| Pentraxin 2/SAP | 4-6 | 1-2 | <1 |
| Pentraxin 3/TSG-14 | <1 | <1 | <1 |
| Periostin/OSF-2 | <1 | <1 | <1 |
| Pref-1/DLK-1/FA1 | 6-10 | 6-10 | <1 |
| Proliferin | 6-10 | 6-10 | <1 |
| Proprotein Convertase | <1 | <1 | 1-2 |
| 9/PCSK9 | | | |
| RAGE | <1 | <1 | <1 |
| RBP4 | 1-2 | 1-2 | <1 |
| Reg3G | 6-10 | 6-10 | <1 |
| Resistin | 2-4 | 2-4 | <1 |
| E-Selectin/CD62E | <1 | <1 | <1 |
| P-Selectin/CD62P | >20 | >10 | <1 |
| Serpin E1/PAI-1 | >10 | 6-10 | <1 |
| Serpin F1/PEDF | <1 | <1 | <1 |
| Thrombopoeitin | <1 | <1 | 1-2 |
| TIM-1/KIM-1/HAVCR | <1 | <1 | 1-2 |
| TNF-α | 2-4 | 1-2 | <1 |
| VCAM-1/CD106 | 4-6 | 6-10 | 1-2 |
| VEGF | <1 | <1 | 1-2 |
| WISP-1/CCN4 | <1 | <1 | <1 |

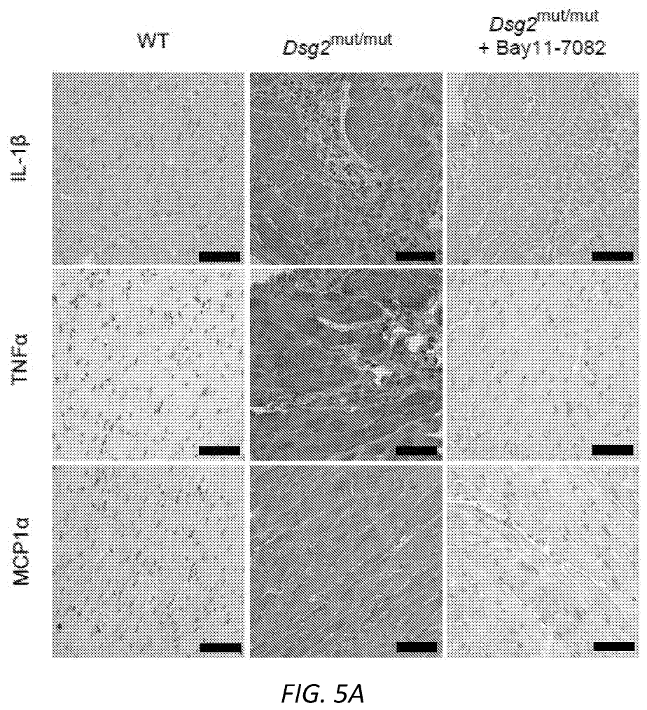

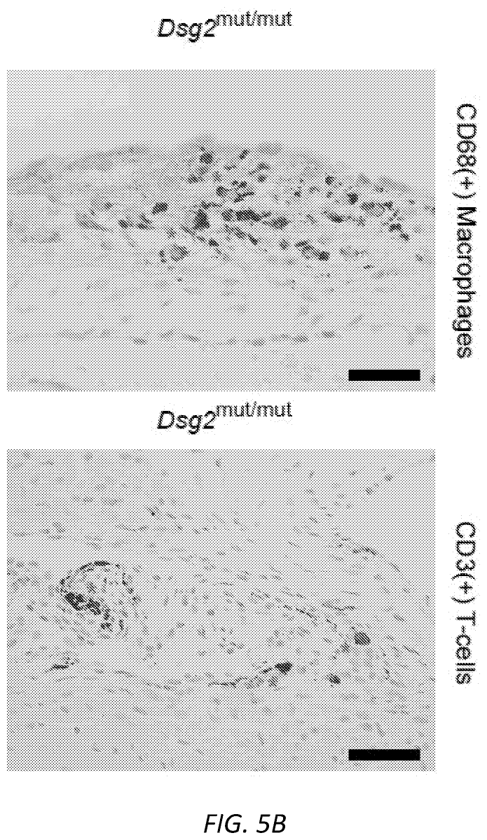

Expression of chemical mediators of the immune response is generally considered to be the province of the professional cells of the adaptive immune system, mainly lymphocytes and macrophages. However, parenchymal cells of most organs, including cardiac myocytes, are capable of producing inflammatory mediators, and we know from the in vitro studies shown in FIG. 2 that neonatal ventricular myocytes that expressed mutant plakoglobin produced and secreted diverse cytokines. To identify the cellular source of cytokines in Dsg2^{mut/mut} mice, we stained sections of myocardium with antibodies against representative key molecules including IL-1β, TNFα and MCP-1α. As shown in FIG. 5, positive immunoreactive signal for IL-1β, TNFα and MCP-1α was apparent in cardiac myocytes and signal for IL-1β and TNFα was seen in infiltrating mononuclear inflammatory cells in Dsg2^{mut/mut} hearts. Signal intensity in both myocytes and inflammatory cells was reduced in mice treated with Bay 11-7082. From the cytokine assays shown in FIG. 4, expression of IL-1β and TNFα in the hearts of Dsg2^{mut/mut} mice suggests a role for monocyte/macrophages and expression of IFNγ suggests a role for T-cells. In fact, both macrophages and T-cells were present in the inflammatory cells infiltrating the hearts of Dsg2^{mut/mut} mice (FIG. 5). Taken together with data in FIG. 2, these observations indicated that inflammation in ACM involves activation of an innate immune response in cardiac myocytes driven, at least in part, by NFκB signaling. Treatment of Dsg2^{mut/mut} mice with Bay 11-7082 not only reduced overall cytokine production (as shown in FIG. 4), but also reduced the total number of infiltrating inflammatory cells in the heart based on a careful survey of myocardial sections.

### Example 4

### Prevention of ACM Disease Features Correlates With Reduction in Cytokine Expression

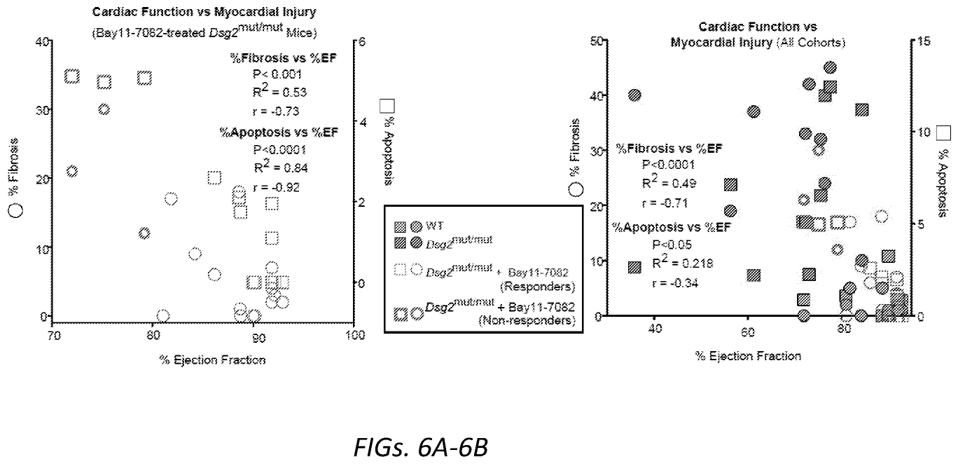

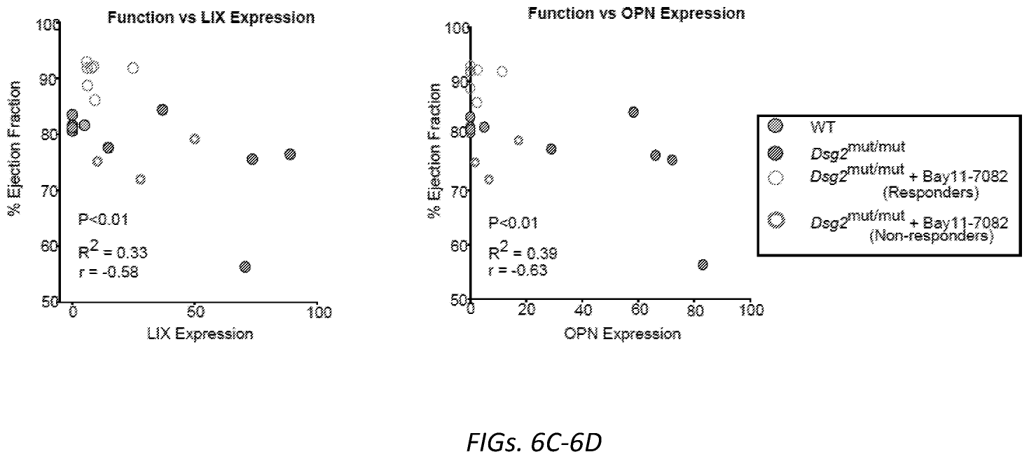

Data shown in FIGS. 3-5 clearly implicate activation of an immune response in the development of the ACM disease phenotype in Dsg2^{mut/mut} mice. However, this does not prove that immune signaling and production of cytokines are directly responsible for driving this phenotype. Nevertheless, we were able to gain further insight into this potential causal relationship by correlating the extent to which Bay 11-7082 mitigated the disease phenotype in individual animals. For example, 3 of 17 treated ACM mice failed to respond to NFκB inhibition. On further investigation, we discovered this was related to a technical failure of drug delivery by implanted Alzet minipumps. As shown in FIG. 6, these 3 mice had worse cardiac function (i.e., lower ejection fractions) than the other Bay11-7082-treated mice in this experiment, in which there was a significant inverse correlation between ejection fraction, the amount of myocardial fibrosis and apoptosis.

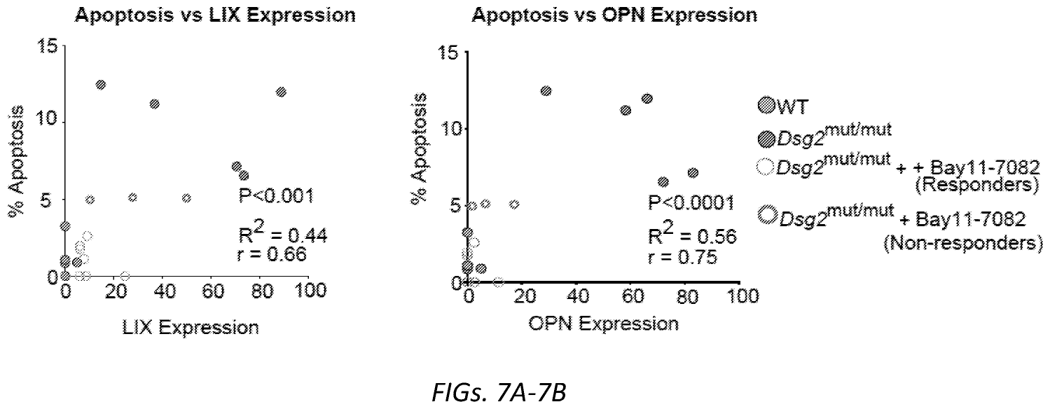

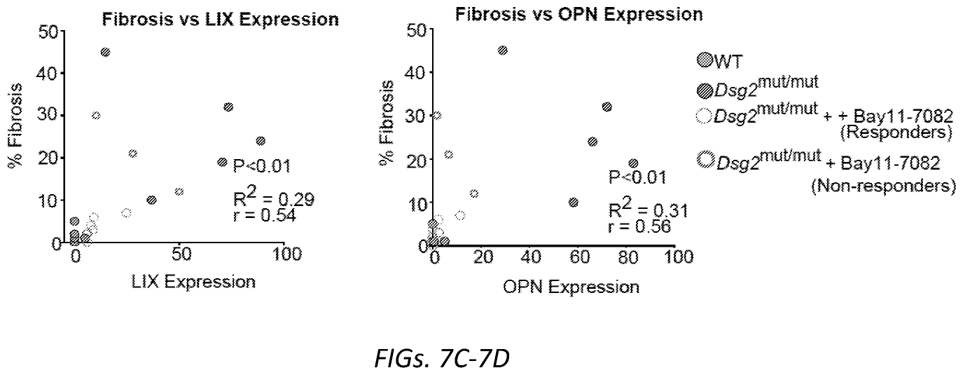

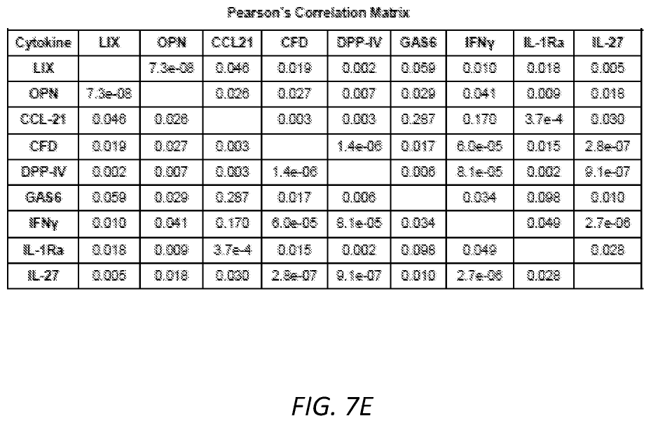

To determine if the amount of expression of any specific cytokine correlated with cardiac function as measured by ejection fraction, we first analyzed all 111 cytokines included in Table 2. Of these, 41 cytokines showed significant up- or down-regulation when comparing WT vs. Dsg2^{mut/mut} mice and/or untreated vs. Bay 11-7082-treated Dsg2^{mut/mut} mice. We then assessed cytokine expression levels in each individual animal to determine if expression of any of these 41 cytokines correlated with cardiac function as measured by ejection fraction. Of these 41 molecules, the levels of LIX (CXCL5) and osteopontin (OPN) both showed a significant inverse correlation with ejection fraction (FIG. 6) and a significant positive correlation with myocardial injury (percent fibrosis and apoptosis) and with each other (FIG. 7). We then used a Pearson's correlation matrix to characterize the relationship between levels of LIX and OPN and the 39 other cytokines reported above.

Levels of LIX (CXCL5) and osteopontin (OPN) both showed a significant inverse correlation with ejection fraction (FIG. 6) and a significant positive correlation with myocardial injury (percent fibrosis and apoptosis) and with each other (FIG. 7). In addition, levels of both LIX and OPN showed significant correlations with 7 other cytokines: CCL21, complement factor D, DPP-IV, GAS6, IFNγ, IL-1Ra and IL-27 (FIG. 7). As discussed below, this network of cytokines regulates fundamental features of the inflammatory response including apoptosis, fibrosis and remodeling of the extracellular matrix. Taken together, these observations clearly implicated NFκB-mediated cytokine production as drivers of the ACM disease phenotype.

### Example 5

### ACM Patient iPSC-Cardiac Myocytes Express Abundant Cytokines Under the Control of NFκB

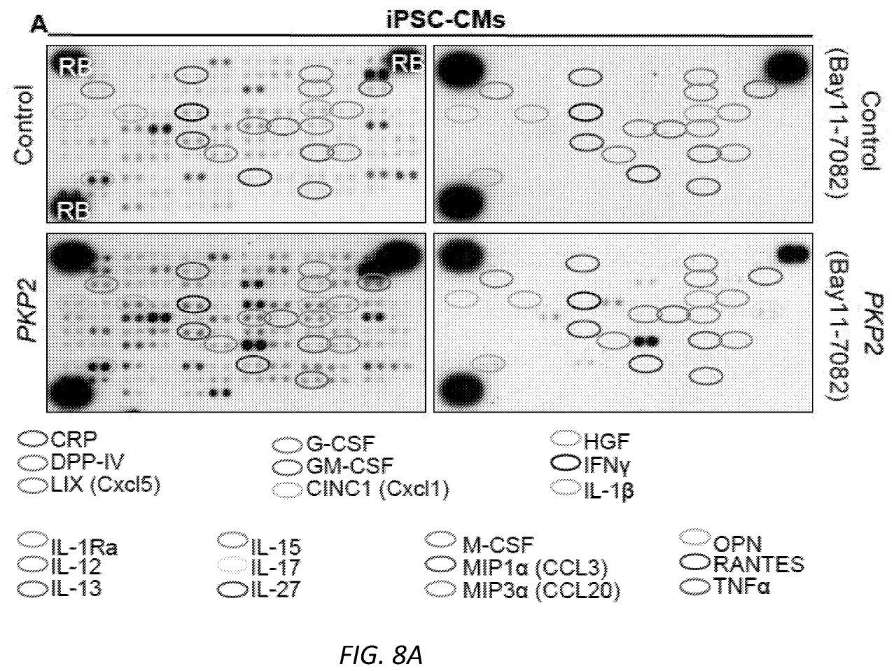

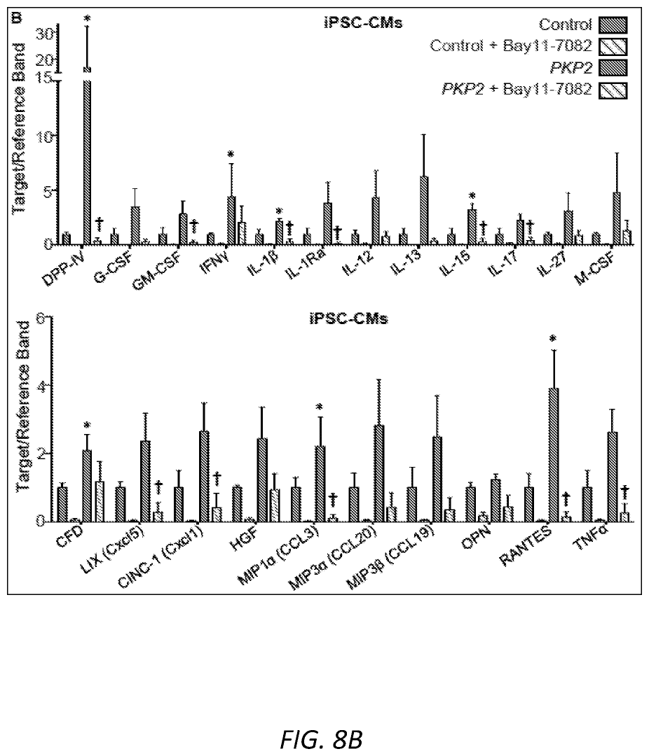

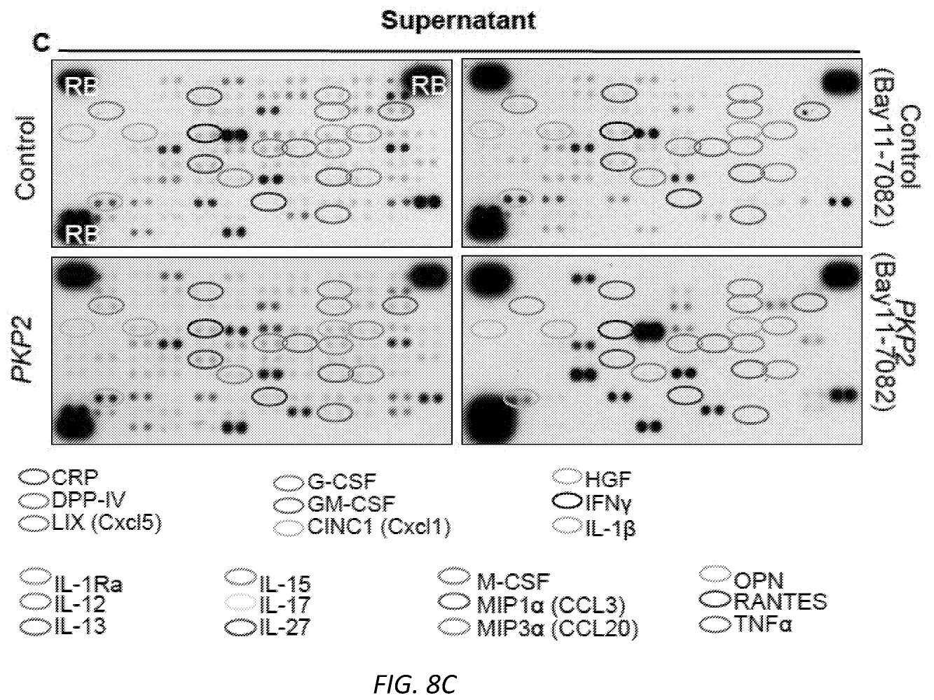

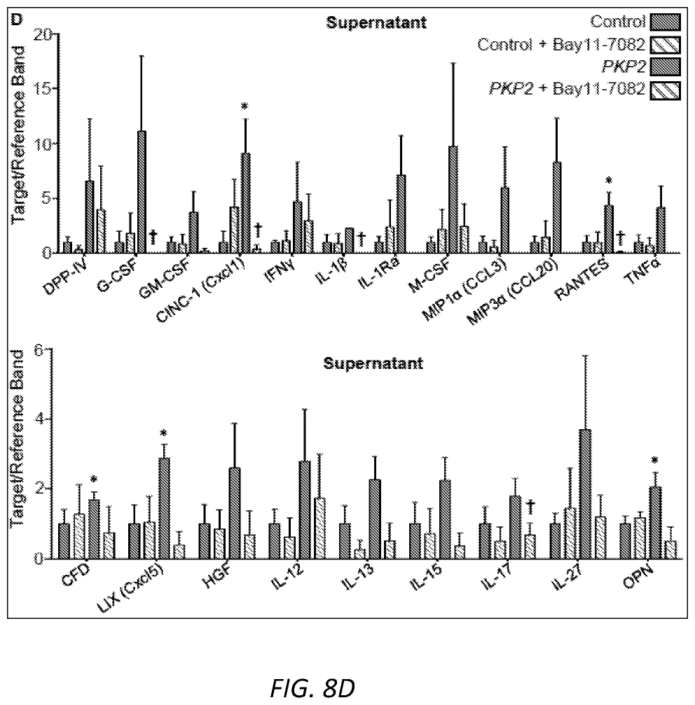

It is not possible to identify and quantify the relative contributions of the innate immune response in cardiac myocytes vs. activation of inflammatory signaling in professional immune cells in studies in Dsg2^{mut/mut} mice presented in FIGS. 3-6. It is likely that both play a role in the pathogenesis of the disease phenotype. However, to gain further insights into the immune response in cardiac myocytes in ACM, and to determine if this occurs as a cell autonomous mechanism in patients with ACM, we characterized cytokine production in cultures of cardiac myocytes derived from hiPSCs obtained from a patient with documented ACM caused by a mutation in the desmosomal gene PKP2. These cultures are composed of more than 95% pure cardiac myocytes and they are devoid of lymphocytes and macrophages. As shown in FIG. 8, when grown under basal conditions in the absence of any provocative stimuli used in previous studies to induce various features of the ACM phenotype,⁴⁹ these cells expressed and secreted into the culture medium large amounts of cytokines including essentially all that were expressed in the hearts of Dsg2^{mut/mut} mice. Furthermore, exposure of ACM patient hiPSC-cardiac myocytes to Bay 11-7082 greatly reduced the amount of cytokines in cells and culture media (FIG. 8). Table 3 shows data for all cytokines measured in control and patient hiPSC-cardiac myocytes and in their culture media. These observations provide additional independent evidence of activation of an innate immune response in cardiac myocytes in ACM under the control of NFκB signaling.

TABLE 3
Cytokine expression levels for in vitro studies in hiPSC cardiac myocytes.

| | | iPSC-CMs | | | | Supernatant | | |
|---|---|---|---|---|---|---|---|---|
| | | PKP2 | PKP2 | Control | | PKP2 | PKP2 | Control |
| | | (Bay11- | (Bay11- | (Bay11- | | (Bay11- | (Bay11- | (Bay11- |
| | PKP2 vs | 7082) vs | 7082) vs | 7082) vs | PKP2 vs | 7082) vs | 7082) vs | 7082) vs |
| Target | Control | Control | PKP2 | Control | Control | Control | PKP2 | Control |
| Adiponectin | >5 | >5 | <2 | 3-5 | <2 | <2 | <2 | <2 |
| Apolipoprotein A-1 | <2 | 2-3 | <2 | <2 | 2-5 | 2-5 | <2 | <2 |
| Angiogenin | >5 | >5 | <2 | <2 | <2 | 2-5 | <2 | <2 |
| Angiopoietin-1 | 3-5 | 2-3 | <2 | <2 | 2-5 | 2-5 | <2 | <2 |
| Angiopoietin-2 | 2-3 | 2-3 | <2 | <2 | <2 | 2-5 | <2 | <2 |
| BAFF/BLyS/TNFSF13B | >5 | 2-3 | <2 | <2 | 2-5 | <2 | <2 | <2 |
| BDNF | >5 | >5 | <2 | <2 | 2-5 | <2 | <2 | <2 |
| Complement Component | 3-5 | <2 | <2 | <2 | 2-5 | <2 | <2 | <2 |
| C5/C5a | | | | | | | | |
| CD14 | 3-5 | <2 | <2 | <2 | <2 | <2 | <2 | <2 |
| CD30 | <2 | <2 | <2 | <2 | <2 | <2 | <2 | <2 |
| CD40 Ligand | 2-3 | <2 | <2 | <2 | 2-5 | <2 | <2 | 2-5 |
| Chitinase 3-like 1 | 2-3 | 2-3 | <2 | <2 | 5-10 | 2-5 | <2 | <2 |
| Complement Factor D | 2-3 | 2-3 | <2 | <2 | <2 | <2 | <2 | <2 |
| C-Reactive Protein/ | 3-5 | 3-5 | <2 | <2 | 2-5 | 2-5 | <2 | <2 |
| CRP | | | | | | | | |
| Cripto-1 | >5 | 2-3 | <2 | <2 | 2-5 | 2-5 | <2 | <2 |
| Cystatin C | >5 | >5 | <2 | <2 | 2-5 | 2-5 | <2 | <2 |
| DKK-1 | 3-5 | 3-5 | <2 | <2 | 2-5 | <2 | <2 | <2 |
| DPPIV/CD26 | >15 | >5 | <2 | <2 | 5-10 | 2-5 | <2 | <2 |
| EGF | 3-5 | <2 | <2 | <2 | 2-5 | <2 | <2 | <2 |
| Emmprin (CD147) | <2 | <2 | <2 | <2 | <2 | <2 | <2 | <2 |
| LIX (Cxcl5) | 2-3 | <2 | <2 | <2 | 2-5 | <2 | <2 | <2 |
| Endoglin/CD105 | <2 | <2 | <2 | <2 | 2-5 | <2 | <2 | <2 |
| Fas Ligand | 2-3 | 3-5 | <2 | <2 | 5-10 | 2-5 | <2 | <2 |
| FGF basic | 2-3 | 2-3 | <2 | <2 | <2 | 2-5 | <2 | <2 |
| FGF-7 | 2-3 | <2 | <2 | <2 | <2 | 2-5 | <2 | <2 |
| FGF-19 | <2 | <2 | <2 | <2 | <2 | <2 | <2 | <2 |
| FLt-3 Ligand | >5 | 2-3 | <2 | <2 | 2-5 | <2 | <2 | <2 |
| G-CSF | 3-5 | <2 | <2 | <2 | >10 | <2 | <2 | <2 |
| GDF-15 | >5 | 2-3 | <2 | <2 | 5-10 | >10 | <2 | <2 |
| GM-CSF | 2-3 | <2 | <2 | <2 | 2-5 | <2 | <2 | <2 |
| GROα (Cxcl1) | 2-3 | <2 | <2 | <2 | 5-10 | 2-5 | <2 | 2-5 |
| GH, Somatotropin | 2-3 | <2 | <2 | <2 | 5-10 | 2-5 | <2 | 2-5 |
| HGF | 2-3 | 2-3 | <2 | <2 | 2-5 | <2 | <2 | <2 |
| ICAM-1 (CD54) | 2-3 | <2 | <2 | <2 | 2-5 | 2-5 | <2 | <2 |
| IFN-y | 3-5 | >5 | 2-3 | <2 | 2-5 | 5-10 | <2 | <2 |
| IGFBP-2 | 3-5 | >5 | 2-3 | <2 | <2 | 2-5 | 2-5 | <2 |
| IGFBP-3 | 3-5 | <2 | <2 | <2 | 2-5 | 2-5 | <2 | <2 |
| IL-1a (IL-1F1) | >5 | >5 | <2 | <2 | 5-10 | >10 | <2 | <2 |
| IL-1β (IL-1F2) | 2-3 | <2 | <2 | <2 | 2-5 | <2 | <2 | <2 |
| IL-1ra (IL-1F3) | 3-5 | <2 | <2 | <2 | 5-10 | <2 | <2 | 2-5 |
| IL-2 | 2-3 | <2 | <2 | <2 | 2-5 | <2 | <2 | <2 |
| IL-3 | 3-5 | <2 | <2 | <2 | 2-5 | <2 | <2 | <2 |
| IL-4 | 2-3 | 2-3 | <2 | <2 | 2-5 | 2-5 | <2 | <2 |
| IL-5 | >15 | >15 | <2 | <2 | 5-10 | >10 | <2 | 2-5 |
| IL-6 | 2-3 | <2 | <2 | <2 | 2-5 | <2 | <2 | <2 |
| IL-8 | <2 | <2 | <2 | <2 | <2 | <2 | <2 | <2 |
| IL-10 | >5 | 3-5 | <2 | <2 | 2-5 | 2-5 | <2 | <2 |
| IL-11 | 2-3 | <2 | <2 | <2 | 2-5 | 2-5 | <2 | <2 |
| IL-12 p70 | 3-5 | 3-5 | <2 | <2 | 2-5 | 2-5 | <2 | <2 |
| IL-13 | >5 | 2-3 | <2 | <2 | 2-5 | <2 | <2 | <2 |
| IL-15 | 3-5 | <2 | <2 | <2 | 2-5 | <2 | <2 | <2 |
| IL-16 | 3-5 | <2 | <2 | <2 | 2-5 | <2 | <2 | <2 |
| IL-17a | 2-3 | <2 | <2 | <2 | <2 | <2 | <2 | <2 |
| IL-18 | >5 | 2-3 | <2 | <2 | 5-10 | 2-5 | <2 | <2 |
| IL-19 | 2-3 | <2 | <2 | <2 | 5-10 | 2-5 | <2 | 2-5 |
| IL-22 | 2-3 | <2 | <2 | <2 | 2-5 | 2-5 | <2 | <2 |
| IL-23 | 3-5 | <2 | <2 | <2 | 5-10 | 2-5 | <2 | <2 |
| IL-24 | 3-5 | 2-3 | <2 | <2 | 2-5 | 2-5 | <2 | <2 |
| IL-27 | 3-5 | 2-3 | <2 | <2 | 2-5 | 2-5 | <2 | <2 |
| IL-31 | 3-5 | 2-3 | <2 | <2 | 5-10 | 2-5 | <2 | <2 |
| IL-32 | >5 | 2-3 | <2 | <2 | 2-5 | 2-5 | <2 | <2 |
| IL-33 | >5 | 2-3 | <2 | <2 | 2-5 | <2 | <2 | <2 |
| IL-34 | 3-5 | <2 | <2 | <2 | 2-5 | <2 | <2 | <2 |
| IP-10 (Cxcl10) | 2-3 | <2 | <2 | <2 | 5-10 | <2 | <2 | <2 |
| I-TAC (Cxcl11) | >5 | <2 | <2 | <2 | >10 | 2-5 | <2 | <2 |
| Kallikrein-3 (PSA) | >5 | >5 | <2 | <2 | 5-10 | 2-5 | <2 | <2 |
| Leptin | <2 | <2 | <2 | <2 | >25 | 2-5 | <2 | <2 |
| LIF | 2-3 | <2 | <2 | <2 | >25 | 2-5 | <2 | <2 |
| Lipocalin-2/NGAL | 2-3 | 2-3 | <2 | <2 | 5-10 | 2-5 | <2 | <2 |
| MCP1 | 2-3 | 2-3 | <2 | <2 | 2-5 | 5-10 | 2-5 | <2 |
| MCP3 | 3-5 | 3-5 | <2 | <2 | 5-10 | 2-5 | <2 | <2 |
| M-CSF | 3-5 | >5 | <2 | <2 | 5-10 | 5-10 | <2 | 2-5 |
| MIF | >5 | 3-5 | <2 | <2 | <2 | 2-5 | <2 | <2 |
| MIG (Cxcl9) | 2-3 | <2 | <2 | <2 | 2-5 | <2 | <2 | <2 |
| MIP-1α/MIP-1β | 2-3 | <2 | <2 | <2 | 5-10 | <2 | <2 | <2 |
| MIP-3α | 2-3 | <2 | <2 | <2 | 5-10 | <2 | <2 | <2 |
| MIP-3β | 2-3 | <2 | <2 | <2 | 2-5 | <2 | <2 | <2 |
| MMP-9 | 3-5 | 2-3 | <2 | <2 | >25 | <2 | <2 | 5-10 |
| Myeloperoxidase | 3-5 | <2 | <2 | <2 | >10 | 2-5 | <2 | <2 |
| Osteopontin | <2 | <2 | <2 | <2 | 2-5 | <2 | <2 | <2 |
| PDGF-AA | <2 | <2 | <2 | <2 | 2-5 | <2 | <2 | <2 |
| PDGF-AB/BB | 2-3 | <2 | <2 | <2 | 5-10 | 2-5 | <2 | <2 |
| Pentraxin 3/TSG-14 | 2-3 | 3-5 | <2 | <2 | <2 | 2-5 | 2-5 | <2 |
| PF4 (Cxcl4) | 3-5 | 2-3 | <2 | <2 | >10 | 5-10 | <2 | <2 |
| RAGE | 3-5 | <2 | <2 | <2 | >10 | 5-10 | <2 | 2-5 |
| RANTES | 3-5 | <2 | <2 | <2 | 2-5 | <2 | <2 | <2 |
| RBP-4 | <2 | <2 | <2 | <2 | 2-5 | <2 | <2 | <2 |
| Relaxin-2 | 2-3 | <2 | <2 | <2 | 5-10 | <2 | <2 | <2 |
| Resistin | <2 | <2 | <2 | <2 | 2-5 | <2 | <2 | <2 |
| SDF-1α (Cxcl12) | <2 | <2 | <2 | <2 | <2 | <2 | <2 | <2 |
| Serpin E1/PAI-1 | <2 | <2 | <2 | <2 | <2 | <2 | <2 | <2 |
| SHBG (ABP) | 2-3 | <2 | <2 | <2 | 2-5 | 2-5 | <2 | <2 |
| ST2 (IL-1 R4) | 2-3 | <2 | <2 | <2 | 5-10 | 5-10 | <2 | 5-10 |
| TARC (CCL17) | 2-3 | <2 | <2 | <2 | 5-10 | 2-5 | <2 | 2-5 |
| TFF3 | >5 | <2 | <2 | <2 | >10 | 5-10 | <2 | 2-5 |
| TfR (CD71) | >5 | >5 | <2 | <2 | >10 | >10 | <2 | 2-5 |
| TGF-α | >5 | <2 | <2 | <2 | 5-10 | <2 | <2 | 2-5 |
| Thrombospondin-1 | >5 | >5 | <2 | <2 | 5-10 | 5-10 | <2 | <2 |
| TNF-α | 2-3 | <2 | <2 | <2 | 2-5 | <2 | <2 | <2 |
| uPAR | 2-3 | <2 | <2 | <2 | 5-10 | <2 | <2 | <2 |
| VEGF | 2-3 | <2 | <2 | <2 | >25 | 5-10 | <2 | 2-5 |
| Vitamin D BP | <2 | <2 | <2 | <2 | 5-10 | 2-5 | <2 | 2-5 |
| CD31 (PECAM-1) | <2 | <2 | <2 | <2 | >25 | 2-5 | <2 | <2 |
| TIM-3 | 3-5 | <2 | <2 | <2 | >25 | 2-5 | <2 | <2 |
| VCAM-1/CD106 | 2-3 | <2 | <2 | <2 | 2-5 | 2-5 | <2 | <2 |

### Example 6

### Inflammatory Cytokines and Treatment With Rapamycin

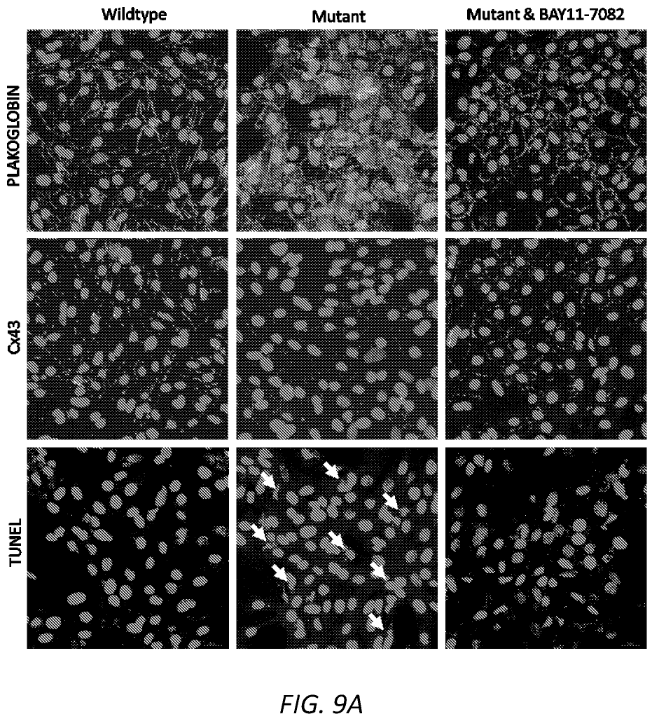

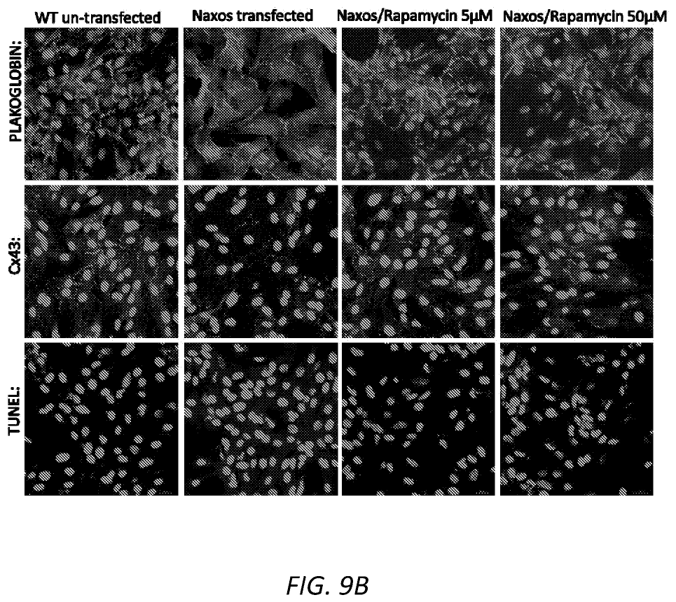

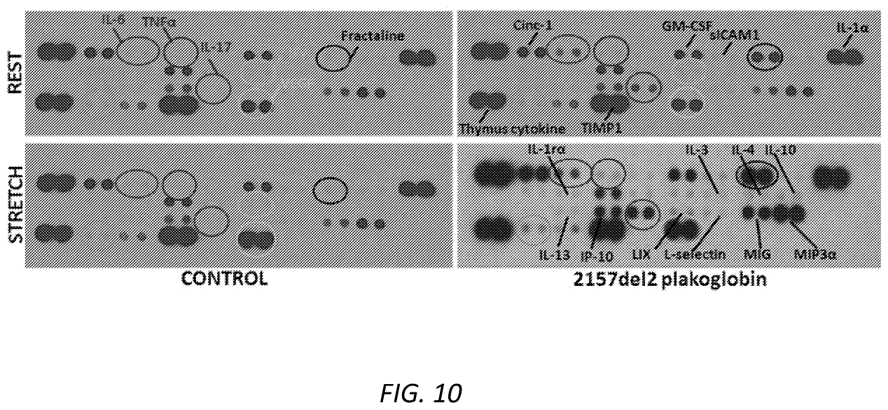

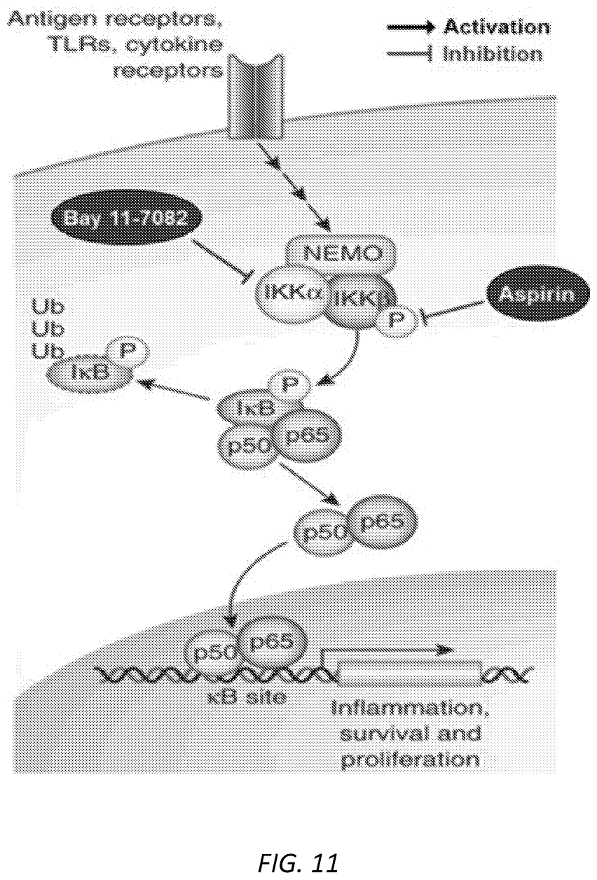

We extensively characterized an in vitro model of ACM involving neonatal rat ventricular myocytes (NRVMs) that express mutant plakoglobin.^{6,7} These cells show key features of ACM seen in patients including redistribution of intercalated disk proteins, myocyte apoptosis and production of inflammatory cytokines.^{6,7} All of these changes are blocked by the GSK3β inhibitor, SB216763.^{6,7} As shown herein, they are also blocked by the NFκB inhibitor Bay 11-7082 (FIG. 9A). In other preliminary in vitro studies, they were also blocked by rapamycin, which inhibits mTOR activation of NFκB signaling downstream of Akt (FIG. 9B). Interestingly, the in vitro readouts of ACM shown in FIG. 9 were greatly increased in cells subjected to cyclical stretch, which we regard as an in vitro surrogate for exercise. In addition, when ACM myocytes were subjected to cyclical stretch, cytokine production (FIG. 10) and apoptosis were greatly increased. By contrast, normal cells responded to stretch by producing VEGF (a physiologic response) but not pro-inflammatory mediators (FIG. 10). These observations suggest a mechanistic link between exercise and inflammation in ACM. This in vitro model is useful for drug screens to identify clinically actionable anti-inflammatory drugs that can mitigate the ACM disease phenotype and prevent exercise-induced sudden death and disease progression. As shown in FIG. 11, Bay 11-7082 blocks NFκB signaling by preventing IκB degradation via direct inhibition of IKKα.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

### REFERENCES

1. Sen-Chowdhry S, Morgan R D, Chambers J C, McKenna W J. Arrhythmogenic cardiomyopathy: etiology, diagnosis, and treatment. Annu Rev Med 2010; 61:233-53.

2. Basso C, Bauce B, Corrado D, Thiene G. Pathophysiology of arrhythmogenic cardiomyopathy. Nat Rev Cardiol 2012; 9:223-33.

3 Corrado D, Link M, Calkins H. Arrhythmogenic right ventricular cardiomyopathy. Now Eng J Med, 2017; 376:61-72

4. James, C A, Bhonsale A, Tichnell C, Murray D. Russell S D, Tandri H, Tedford R J, Judge D P, Calkins H. Exercise increases age-related penetrance and arrhythmic risk in arrhythmogenic right ventricular dysplasia/cardiomyopathy-associated desmosomal mutation carriers. J Am Coll Cardiol 2013; 62:1290-1297.

5. Rojas A, Calkins H. Present understanding of the relationship between exercise and arrhythmogenic right ventricular dysplasia/cardiomyopathy. Trends Cardiovasc Med 2015; 25:181-188.

6. Chelko S P, Asimaki A, Andersen P, Bedja D, Amat-Alarcon N, DeMazumder D, Jasti R, MacRae C A, Leber R, Kleber A G, Saffitz J E, Judge DP. Central role for GSK3β in the pathogenesis of arrhythmogenic cardiomyopathy. JCI Insight 2016:1(5). pii: e85923.

7. Asimaki A, Kapoor S, Plovie E, Karin Arndt A, Adams E, Liu Z, James C A, Judge D P, Calkins H, Churko J, Wu J C, MacRae C A, Kléber A G, Saffitz J E. Identification of a new modulator of the intercalated disc in a zebrafish model of arrhythmogenic cardiomyopathy. Sci Transl Med 2014; 6:240ra74.

8. Marcus F I, Fontaine G H, Guiraudon G, Frank R, Laurenceau J L, Malergue C, Grogogeat Y. Right ventricular dysplasia: a report of 24 adult cases. Circulation 1982; 65:384-398.

9. Fontaine G, Fontaliran F, Andrade F R, Velasquez E, Tonet J, Jouven X, Fujioka Y, Frank R. The arrhythmogenic right ventricle. Dysplasia versus cardiomyopathy. Heart Vessels 1995; 10:227-235.

10. Corrado D, Basso C, Thiene G, McKenna W J, Davies M J, Fontaliran F, Nava A, Silvestri F, Blomstrom-Lundqvist C, Wlodarska E K, Fontaine G, Camerini F. Spectrum of clinicopathologic manifestations of arrhythmogenic right ventricular cardiomyopathy/dysplasia: a multicenter study. J Am Coll Cardiol 1997; 30:1512-1520.

11. Lopez-Ayala J M, Pastor-Quirante F, Gonzalez-Carrillo J, Lopez-Cuenca D, Sanchez-Munoz J J, Oliva-Sandoval J R, Gimeno J R. Genetics of myocarditis in arrhythmogenic right ventricular dysplasia. Heart Rhythm 2015; 12:766-773.

12. Asimaki A, Tandri H, Duffy E R, Winterfield J R, Mackey-Bojack S, Picken M M, Cooper L T, Wilber D J, Marcus F I, Basso C, Thiene G, Tsatsopoulou A, Protonotarios N, Stevenson W G, McKenna W J, Gautam S, Remick D G, Calkins, Saffitz J E. Altered desmosomal proteins in granulomatous myocarditis and potential pathogenic links to arrhythmogenic right ventricular cardiomyopathy. Circ Arrhyth Electrophysiol 2011; 4:743-52.

13. Mendell J R, Moxley R T, Griggs R C, Brooke M H, Fenichel G M, Miller J P, King W, Signore K, Pandya S, Florence J, et al. Randomized, double-blind six-month trial of prednisone in Duchenne's muscular dystrophy. N Eng J Med 1989; 320:1592-1597.

14. Markham L W, Kinnett K, Wong B L, Benson W D, Cripe L H. Corticosteroid treatment retards development of ventricular dysfunction in Duchenne muscular dystrophy. Neuromuscul Disord 2008; 18:365-370.

15. Coghlan M P, Culbert A A, Cross D A, Corcoran S L, Yates J W, Pearce N J, Rausch O L, Murphy G J, Carter P S, Roxbee Cox K. Mills D, Brown M J, Haigh D, Ward R W, Smith D G, Murray K J, Reith A D, Holder J C. Selective small molecule inhibitors of glycogen synthase kinase-3 modulate glycogen metabolism and gene transcription. Chem Biol 2000; 7:793-803.

16. Asimaki A, Tandri J, Huang H, Halushka M K, Gautam S, Basso C, Thiene G, Tsatsopoulou A, Protonotarios N, McKenna W J, Calkins H, Saffitz J E. A new diagnostic test for arrhythmogenic right ventricular cardiomyopathy. New Eng J Med 2009:360:1075-84.

17. Garcia-Gras E, Lombardi R, Giocondo M R, Willerson J T, Schneider M D, Khoury D S, Marian A J. Suppression of canonical Wnt/beta-catenin signaling by nuclear plakoglobin recapitulates phenotype of arrhythmogenic right ventricular cardiomyopathy. J Clin Invest 2006; 116:2012-21. PMCID: 1483165

18. Lombardi R, da Graca Cabreira-Hansen M, Bell A, Fromm R R, Willerson J T, Marian A J. Nuclear plakoglobin is essential for differentation of cardiac progenitor cells to adipocytes in arrhythmogenic right ventricular cardiomyopathy. Circ Res 2011; 109:1342-53.

19. Perkins N D. Post-translational modifications regulating the activity and function of the nuclear factor kappa B pathway. Oncogene 2006; 25:6717-6730.

20. Hayden M S, Ghosh S. Shared principles in NF-kappaB signaling. Cell 2008; 132:344-362.

21. Takada Y, Fang X, Jamaluddin M S, Boyd D D, Aggarwal B B. Genetic deletion of glycogen synthase kinase-3beta abrogates activation of IkappaBalpha kinase, JNK, Akt, and p44/p42 MAPK but potentiates apoptosis induced by tumor necrosis factor. J Biol Chem 2004; 279:39541-39554.

22. Fiol C J, Williams J S, Chou C H, Wang Q M, Roach P J, Andrisani O M. A secondary phosphorylation of CREB341 at Ser129 is required for the cAMP-mediated control of gene expression. A role for glycogen synthase kinase-3 in the control of gene expression. J Biol Chem 1994; 269:32187-32193.

23. Bullock B P, Habener J F. Phosphorylation of the cAMP response element binding protein CREB by cAMP-dependent protein kinase A and glycogen synthase kinase-3 alters DNA binding affinity, conformation, and increases net charge. Biochemistry 1998; 37:3795-3809.

24. Martin M, Rehani K, Jope R S, Michalek S M. Toll-like receptor-mediated cytokine production is differentially regulated by glycogen synthase kinase 3. Nat Immunol 2005; 6:777-784.

25. Rigato I, Bauce B, Rampazzo A, Zorzi A, Pilichou K, Mazzotti E, Migliore F, Marra M P, Lorenzon A, De Bortoli M, Calore M, Nava A, Daliento L, Gregori D, Iliceto S, Thiene G, Basso C, Corrado D. Compound and digenic heterozygosity predicts lifetime arrhythmic outcome and sudden cardiac death in desmosomal gene-related arrhythmogenic right ventricular cardiomyopathy. Circ Cardiovasc Genet 2013; 6:533-542.

26. Bhonsale A, Groeneweg J A, James C A, Dooijes D, Tichnell C Jongbloed J D, Murray B, to Riele A S, van den Berg M P, Bikker H, Atsma D E, de Groot N M, Houweling A C, van der Heijden J F, Russel S D, Doevendans P A, van Veen T A, Tandri H, Wilde A A, Judge D P, van Tintelen J P, Calkins H, Hauer R N. Impact of genotype on clinical course in arrhythmogenic right ventricular dysplasia/cardiomyopathy-associated mutation carriers. Eur Heart J 2015; 36:847-855.

27. Misra A, Haudek S B, Knuefermann P, Vallejo J G, Chen Z J, Michael L H, Sivasubramanian N, Olson E N, Entman M L, Mann D L. Nuclear factor-κB protects the adult cardiac myocyte against ischemia-induced apoptosis in a murine model of acute myocardial infarction. Circulation 2003; 108:3075-3078.

28. Ushach I, Zlotnik A. Biological role of granulocyte macrophage colony-stimulating factor (GM-CSF) and macrophage colony-stimulating factor (M-CSF) on cells of the myeloid lineage. J Leukoc Biol 2016; 100:481-489.

29. Wicks I P, Roberts A W. Targeting GM-CSF in inflammatory diseases. Nat Rev Rheumatol 2016; 12:37-48.

30. Dranoff G, Mulligan R C. Activites of granulocyte-macrophage colony-stimulating factor revealed by gene transfer and gene knockout studies. Stem Cells 1994; Supple 1:173-182.

31. Hamilton J A. GM-CSF as a target in inflammatory/autoimmune disease: current evidence and future therapeutic potential. Expert Rev Clin Immunol 2015; 11:457-465.

32. Anzai A, Choi J L, He S, Fenn A M, Nairz M, Rattik S, McAlpine C S, Mindur J E, Chan C T, Iwamoto Y, Tricot B, Wojtkiewicz G R, Weissleder R, Libby P, Nahrendorf M, Stone J R, Becher B, Swirski F K. The infarcted myocardium solicits GM-CSF for the detrimental oversupply of inflammatory leukocytes. J Exp Med 2017; 214:3293-3310.

33. Harada M, Van Wagoner D R, Nattel S. Role of inflammation in atrial fibrillation: pathophysiology and management. Circ J 2015;79:495-502.

34. Dugo L, Collin M, Thiemermann C. Glycogen synthase kinase 3β as a target for the therapy of shock and inflammation. Shock 2007; 27:113-123.

35. Klamer G, Song E, Ko KH, O'Brien T A, Dolnikov A. Using small molecule GSK3β inhibitors to treat inflammation. Curr Med Chem 2010; 17:2873-2881.

36. Ridker P R, Everett B M, Thuren T, MacFayden J G, Chang W H, Ballantyne C, Forseca F, Nicolau J, Koenig W, Anker S D, Kastelein J J P, Cornel J H, Pais P, Pella D, Genest J, Cifkova R, Lorenzatti A, Forster T, Kobalava Z, Vida-Simiti L, Flather M, Shimokawa H, Ogawa H, Dellborg M, Rossi P R F, Troquay R P T, Libby P, Glynn R J, CANTOS trial group. Anti-inflammatory therapy with cankinumab for atherosclerotic disease. N Eng J Med 2017; 377:1119-1131.

37. MacRae C A, Taylor M R, Mestroni L, Moses J R, Ashley E A, Wheeler M T, Lakdawala N K, Hershberger R E, Ptaszynski M, Sandor V, Saunders M E, Oliver C, Lee P A, Judge D P. Phase 2 study of A797, an oral, selective p38 mitogen-activated protein kinase inhibitor, in patients with Lamin A/C-related dilated cardiomyopathy. e-poster, European Society of Cardiology Congress, August 27-31, Rome, Italy.

38. Sakai N, Wada T, Furuichi K, Iwata Y, Yoshimoto K, Kitagawa K, Kokubo S, Kobayashi M, Takeda S, Kida H, Kobayashi K, Mukaida N, Matsushima K, Yokoyama H. p38 MAPK phosphorylation and NF-κB activation in human crescentic glomerulonephritis. Nephrol Dial Transplant 2002; 17:998-1004.

39. Luo S F, Lin C C Chen H C, Lin W N, Lee I T, Lee C W, Hsiao L D, Yang C M. Involvement of MAPKs, NF-kappaB and p300 co-activator in IL-1beta-induced cytosolic phospholipase A2 expression in canine tracheal smooth muscle cells. Toxicol Appl Pharmacol 2008; 232:396-407.

40. Vercammen E, Staal J, Van Den Broeke A, Haegman M, Vereecke L, Schotte P, Beyaert R. Prolonged exposure to IL-1beta and IFNgamma induces necrosis of L929 tumor cells via a p38MAPK-NKkappaB/NO-dependent mechanism. Oncogene 2008; 27:3780-3788.

41. Kopp W, Ghosh S. Inhibition of NF-kappa B by sodium salicylate and aspirin. Science 1994; 265:956-959.

42. Yuan M, Konstantopoulos N, Lee J, Hansen L, Li Z W, Karin M, Shoelson S E. Reversal of obesity- and diet-induced insulin resistance with salicylates or targeted disruption of Ikkbeta. Science 2001; 293:1673-1677.

43. Amann R, Peskar B A. Anti-inflammatory effects of aspirin and sodium salicylate. Eur J Pharmacol 2002:447-1-9.

44. Hariharan V, Asimaki A, Michaelson J E, Plovie E, MacRae C A, Saffitz J E, Huang H. Arrhythmogenic right ventricular cardiomyopathy mutations alter shear response without changes in cell-cell adhesion. Cardiovasc Res 2014; 104:280-289.

45. Asimaki A, Protonotarios A, James C A, Chelko S P, Tichnell C, Murray B, Tsatsopoulou A, Anastasakis A, to Riele A, Kleber A G, Judge D P, Calkins H, Saffitz J E: Characterizing the molecular pathology of arrhythmogenic cardiomyopathy in patient buccal mucosa cells. Circ Arrhythm Electrophysiol. 2016; 9: e003688.

46. Zhan T, Rindtorff N, Boutros M: Wnt signaling in cancer. Oncogene. 2017; 36:1461-1473.

47. Campbell J J, Perkins N D. Regulation of NF-kappaB function. Biochem Soc Symp. 2006; 73:165180.

48. Syed F, Diwan A, Hahn H S. Murine echocardiography: a practical approach for phenotyping genetically manipulated and surgically modeled mice. J Am Soc Echocardiogr. 2005; 18:982-990.

49. Kim C, Wong J, Wen J, Wang S, Wang C, Spiering S, Kan N G, Forcales S, Puri P L, Leone T C, Marine J E, Calkins H, Kelly D P, Judge D P, Chen H S. Studying arrhythmogenic right ventricular dysplasia with patient-specific iPSCs. Nature. 2013; 494:105-110.

50. Bhattacharya S, Burridge P W, Kropp E M, Chuppa S L, Kwok W M, Wu J C, Boheler K R, Gundry R L. High efficiency differentiation of human pluripotent stem cells to cardiomyocytes and characterization by flow cytometry. J Vis Exp. 2014; 91:52014.

51. McKoy Protonotarios N, Crosby A, Tsatsoupoulou A, Anastasakis A, Coonar A, Norman M, Baboonian C, Jeffery S, McKenna W J. Identification of a deletion in plakoglobin in arrhythmogenic right ventricular cardiomyopathy with palmoplantar keratoderma and woolly hair (Naxos disease). Lancet. 2000; 355:2119-2124.

52. Basso C, Thiene G, Corrado D, Angelini A, Nava A, Valente M. Arrhythmogenic right ventricular cardiomyopathy. Dysplasia, dystrophy or myocarditis? Circulation. 1996; 94:983-991.

53. Lazzerini P E, Capecchi P L, Laghi-Pasini F. Systemic inflammation and arrhythmic risk: lessons from rheumatoid arthritis. Eur Heart J. 2017; 38:1717-1727.

54. Miller S C, Huang R, Sakamuru S, Shukla S J, Attene-Ramos M S, Shinn P, Van Leer D, Leister W, Austin C P, Xia M. Identificatioin of known drugs that act as inhibitors of NF-kappaB signaling and their mechanism of action. Biochem Pharmacol. 2010; 79:1272-1280.

1. A method of treating a subject with arrhythmogenic cardiomyopathy (ACM), the method comprising:
identifying a subject as having or at risk of developing ACM; and
administering to the subject a therapeutically effective amount of an inhibitor of NFκB signaling.

2. The method of claim 1, wherein the method further comprises one or more of recommending or advising the subject to avoid strenuous or intense physical activity or exercise; recommending or prescribing or administering one or more Singh Vaughan Williams class II antiarryhthmics (beta blockers) such as propranolol, esmolol, timolol, metoprolol, or atenolol; recommending or prescribing or administering one or more class III anti-arrhythmics (K-channel blockers) such as amiodarone, sotalol, ibutilide, dofetilide, dronedarone or E-4031;
recommending or performing cardiac ablation; or recommending or implanting an implantable cardiac defibrillator (ICD).

3. The method of any of claim 1, wherein the inhibitor of NFκB signaling is selected from the group consisting of DNA binding inhibitors that inhibit the binding between NFκB and DNA; inhibitors of post-translational modifications on NFκB including a p65 acetylation inhibitor; translocation inhibitors that prevents NFκB from translocating to the nucleus; IκB degradation inhibitors that prevents ubiquitinated IκB from being degraded; IKK inhibitors that prevent the phosphorylation of IκB bound to NFκB.

4. The method of claim 3, wherein the inhibitor of NFκB signaling is an IKK inhibitor that prevents the phosphorylation of IκB bound to NFκB.

5. The method of claim 4, wherein the IKK inhibitor is an ATP analog, an allosteric modulator, or an agent interfering with the kinase activation loops.

6. The method of claim 5, wherein the IKK inhibitor is selected from the group consisting of β-carboline, SPC-839, BMS-345541, SAR-113945, and Bay 11-7082.

7. The method of claim 1, wherein the inhibitor of NFκB signaling is selected from the group consisting of Bay 11-7082; Bithionol; Bortezomib; Cantharidin; Chromomycin A3; Daunorubicinum; Digitoxin; Ectinascidin 743; Emetine; Fluorosalan; Manidipine hydrochloride; Narasin; Lestaurtinib; Ouabain; Rapamycin; Sorafenib tosylate; Sunitinib malate; Tioconazole; Tribromsalan; Triclabendazolum; and Zafirlukast.

8. The method of claim 7, wherein the inhibitor of NFκB signaling is Bay 11-7082.

9. The method of claim 7, wherein the inhibitor of NFκB signaling is rapamycin.

10-18. (canceled)
